(19) United States
(12) Patent Application Publication
(10) Pub. No.: US 2023/0389806 A1
(43) Pub. Date: Dec. 7, 2023

(54) USER INTERFACES RELATED TO PHYSIOLOGICAL MEASUREMENTS

(71) Applicant: Apple Inc., Cupertino, CA (US)

(72) Inventors: Nicholas D. FELTON, Sunnyvale, CA (US); Alexander DICKINSON, San Francisco, CA (US); Eamon F. GILRAVI, San Francisco, CA (US); Katherine NIEHAUS, San Francisco, CA (US); William R. POWERS, III, San Francisco, CA (US); Adeeti V. ULLAL, Los Altos, CA (US)

(21) Appl. No.: 17/982,968

(22) Filed: Nov. 8, 2022

Related U.S. Application Data
(60) Provisional application No. 63/348,938, filed on Jun. 3, 2022.

(51) Int. Cl. A61B 5/0205; G06F 3/01; A61B 5/00; A63B 24/00

(52) U.S. Cl. CPC A61B 5/0205; G06F 3/011; A61B 5/7275; A61B 5/742; A63B 24/0062; G06F 3/0482

(57) ABSTRACT

Detecting a physiological parameter of a user at a first level during a first activity and at a second level during a second activity and displaying, based on the first level and the second level, a predictive change in the physiological parameter had the second activity been a third activity that is different from the second activity.

### CROSS-REFERENCE TO RELATED APPLICATIONS

This application claims priority to U.S. Provisional Application No. 63/348,938, filed Jun. 3, 2022, the entire contents of which are hereby incorporated by reference.

### FIELD

The present disclosure relates generally to computer user interfaces, and more specifically to techniques for displaying user interfaces including information related to physiological measurements.

### BACKGROUND

Personal electronic devices allow users to view information related to physiological measurements. Some personal electronic devices include the ability to collect users' physiological measurements. Some personal electronic devices include the ability to display user interfaces related to physiological measurements.

### BRIEF SUMMARY

Some techniques for displaying user interfaces including information related to physiological measurements using electronic devices, however, are generally cumbersome and inefficient. For example, some existing techniques use a complex and time-consuming user interface, which may include multiple key presses or keystrokes. Existing techniques require more time than necessary, wasting user time and device energy. This latter consideration is particularly important in battery-operated devices.

Accordingly, the present technique provides electronic devices with faster, more efficient methods and interfaces for displaying user interfaces including information related to physiological measurements. Such methods and interfaces optionally complement or replace other methods for displaying user interfaces including information related to physiological measurements. Such methods and interfaces reduce the cognitive burden on a user and produce a more efficient human-machine interface. For battery-operated computing devices, such methods and interfaces conserve power and increase the time between battery charges.

In accordance with some embodiments, a method, performed at a computer system that is in communication with a display generation component and one or more sensors is described. The method comprises: while a user of the computer system performs a first activity, detecting, via the one or more sensors, that a physiological parameter of the user of the computer system is at a first level; subsequent to detecting the physiological parameter at the first level and while the user of the computer system performs a second activity that is different from the first activity, detecting, via the one or more sensors, that the physiological parameter of the user of the computer system is at a second level that is different from the first level; and displaying, via the display generation component and based on the first level and the second level, a predictive change in the physiological parameter had the second activity been a third activity that is different from the second activity.

In accordance with some embodiments, a non-transitory computer-readable storage medium is described. The non-transitory computer-readable storage medium stores one or more programs configured to be executed by one or more processors of a computer system that is in communication with a display generation component and one or more sensors, the one or more programs including instructions for: while a user of the computer system performs a first activity, detecting, via the one or more sensors, that a physiological parameter of the user of the computer system is at a first level; subsequent to detecting the physiological parameter at the first level and while the user of the computer system performs a second activity that is different from the first activity, detecting, via the one or more sensors, that the physiological parameter of the user of the computer system is at a second level that is different from the first level; and displaying, via the display generation component and based on the first level and the second level, a predictive change in the physiological parameter had the second activity been a third activity that is different from the second activity.

In accordance with some embodiments, a transitory computer-readable storage medium is described. The transitory computer-readable storage medium stores one or more programs configured to be executed by one or more processors of a computer system that is in communication with a display generation component and one or more sensors, the one or more programs including instructions for: while a user of the computer system performs a first activity, detecting, via the one or more sensors, that a physiological parameter of the user of the computer system is at a first level; subsequent to detecting the physiological parameter at the first level and while the user of the computer system performs a second activity that is different from the first activity, detecting, via the one or more sensors, that the physiological parameter of the user of the computer system is at a second level that is different from the first level; and displaying, via the display generation component and based on the first level and the second level, a predictive change in the physiological parameter had the second activity been a third activity that is different from the second activity.

In accordance with some embodiments, a computer system that is configured to communicate with a display generation component and one or more sensors is described. The computer system comprises: one or more processors; and memory storing one or more programs configured to be executed by the one or more processors, the one or more programs including instructions for: while a user of the computer system performs a first activity, detecting, via the one or more sensors, that a physiological parameter of the user of the computer system is at a first level; subsequent to detecting the physiological parameter at the first level and while the user of the computer system performs a second activity that is different from the first activity, detecting, via the one or more sensors, that the physiological parameter of the user of the computer system is at a second level that is different from the first level; and displaying, via the display generation component and based on the first level and the second level, a predictive change in the physiological parameter had the second activity been a third activity that is different from the second activity.

In accordance with some embodiments, a computer system that is configured to communicate with a display generation component and one or more sensors is described. The computer system comprises: while a user of the computer system performs a first activity, means for detecting, via the one or more sensors, that a physiological parameter of the user of the computer system is at a first level; subsequent to detecting the physiological parameter at the first level and while the user of the computer system performs a second activity that is different from the first activity, means for detecting, via the one or more sensors, that the physiological parameter of the user of the computer system is at a second level that is different from the first level; and means for displaying, via the display generation component and based on the first level and the second level, a predictive change in the physiological parameter had the second activity been a third activity that is different from the second activity.

In accordance with some embodiments, a computer program product is described. The computer program product comprises one or more programs configured to be executed by one or more processors of a computer system that is in communication with a display generation component and one or more sensors. The one or more programs include instructions for: while a user of the computer system performs a first activity, detecting, via the one or more sensors, that a physiological parameter of the user of the computer system is at a first level; subsequent to detecting the physiological parameter at the first level and while the user of the computer system performs a second activity that is different from the first activity, detecting, via the one or more sensors, that the physiological parameter of the user of the computer system is at a second level that is different from the first level; and displaying, via the display generation component and based on the first level and the second level, a predictive change in the physiological parameter had the second activity been a third activity that is different from the second activity.

Executable instructions for performing these functions are, optionally, included in a non-transitory computer-readable storage medium or other computer program product configured for execution by one or more processors. Executable instructions for performing these functions are, optionally, included in a transitory computer-readable storage medium or other computer program product configured for execution by one or more processors.

Thus, devices are provided with faster, more efficient methods and interfaces for displaying user interfaces including information related to physiological measurements, thereby increasing the effectiveness, efficiency, and user satisfaction with such devices. Such methods and interfaces may complement or replace other methods for displaying user interfaces including information related to physiological measurements.

### DESCRIPTION OF THE FIGURES

For a better understanding of the various described embodiments, reference should be made to the Description of Embodiments below, in conjunction with the following drawings in which like reference numerals refer to corresponding parts throughout the figures.

FIG. 1A is a block diagram illustrating a portable multifunction device with a touch-sensitive display in accordance with some embodiments.

FIG. 1B is a block diagram illustrating exemplary components for event handling in accordance with some embodiments.

FIG. 2 illustrates a portable multifunction device having a touch screen in accordance with some embodiments.

FIG. 3 is a block diagram of an exemplary multifunction device with a display and a touch-sensitive surface in accordance with some embodiments.

FIG. 4A illustrates an exemplary user interface for a menu of applications on a portable multifunction device in accordance with some embodiments.

FIG. 4B illustrates an exemplary user interface for a multifunction device with a touch-sensitive surface that is separate from the display in accordance with some embodiments.

FIG. 5A illustrates a personal electronic device in accordance with some embodiments.

FIG. 5B is a block diagram illustrating a personal electronic device in accordance with some embodiments.

FIGS. 6A-6M illustrate exemplary devices and user interfaces for displaying user interfaces including information related to physiological measurements, in accordance with some embodiments.

FIG. 7 is a flow diagram illustrating a method for displaying user interfaces including information related to physiological measurements, in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for electronic devices that provide efficient methods and interfaces for displaying user interfaces including information related to physiological measurements. For example, there is a need for displaying a user's physiological parameters and predictive changes in such parameters to a user. Such techniques can reduce the cognitive burden on a user who accesses user interfaces including information related to physiological measurements, thereby enhancing productivity. Further, such techniques can reduce processor and battery power otherwise wasted on redundant user inputs.

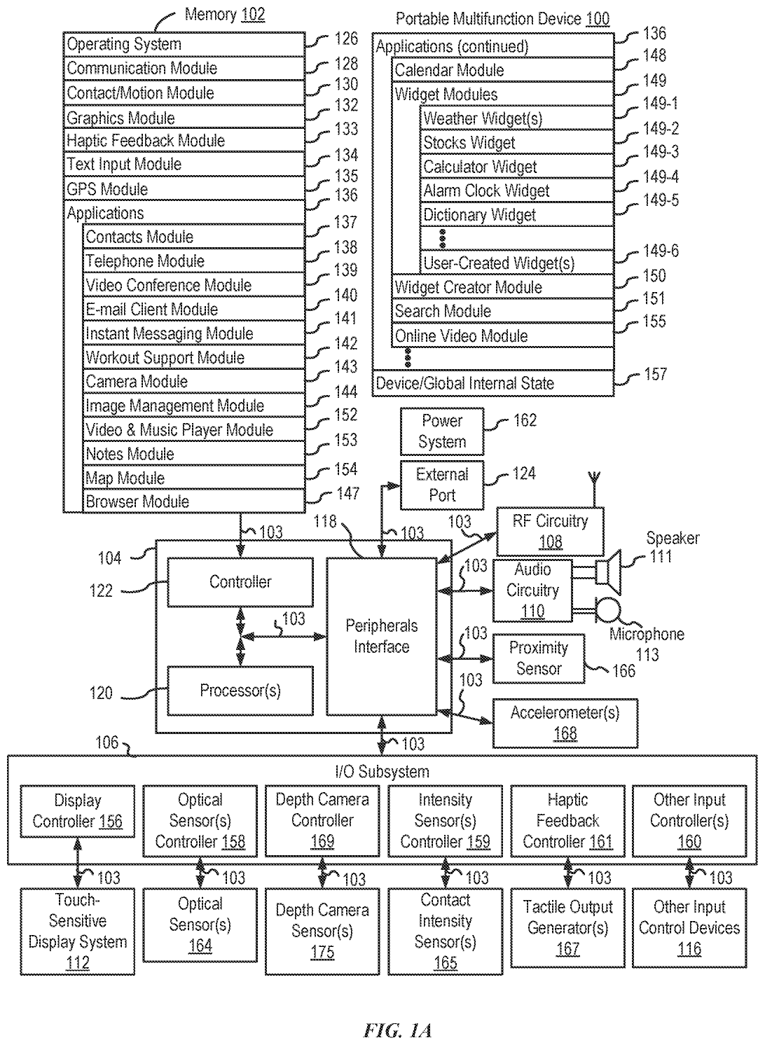

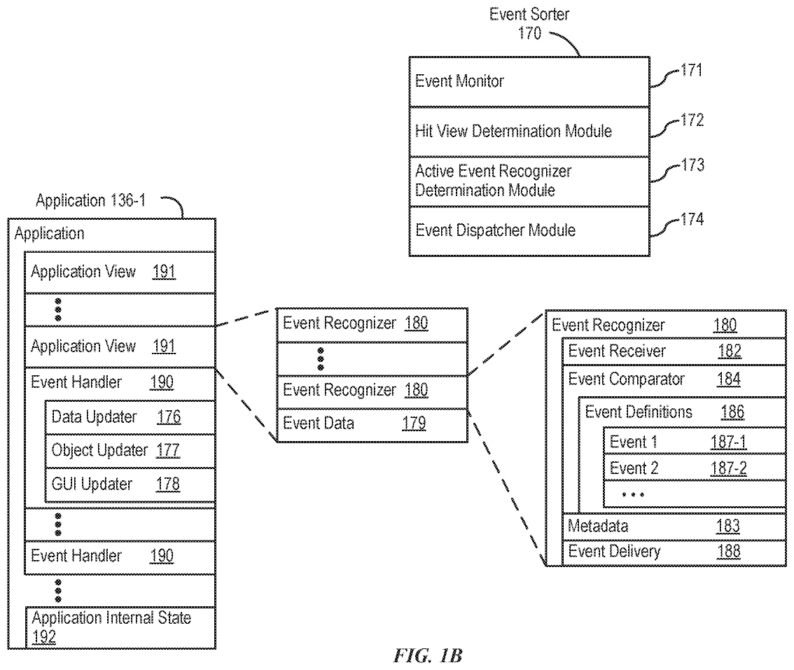

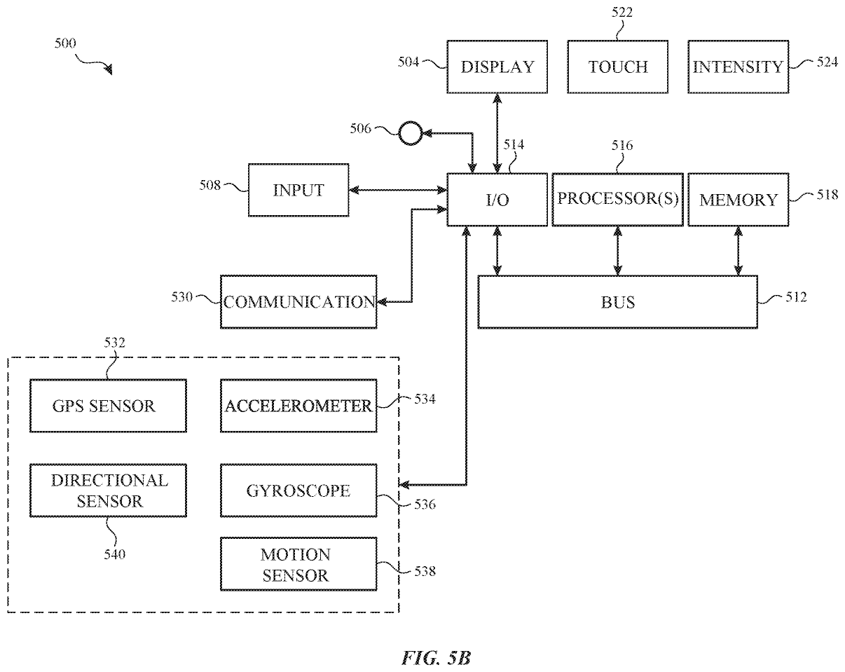

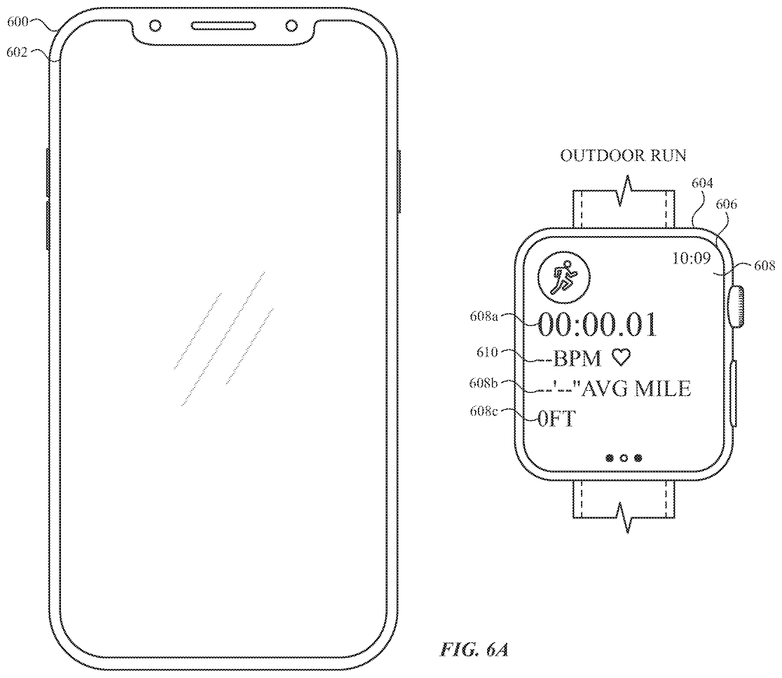

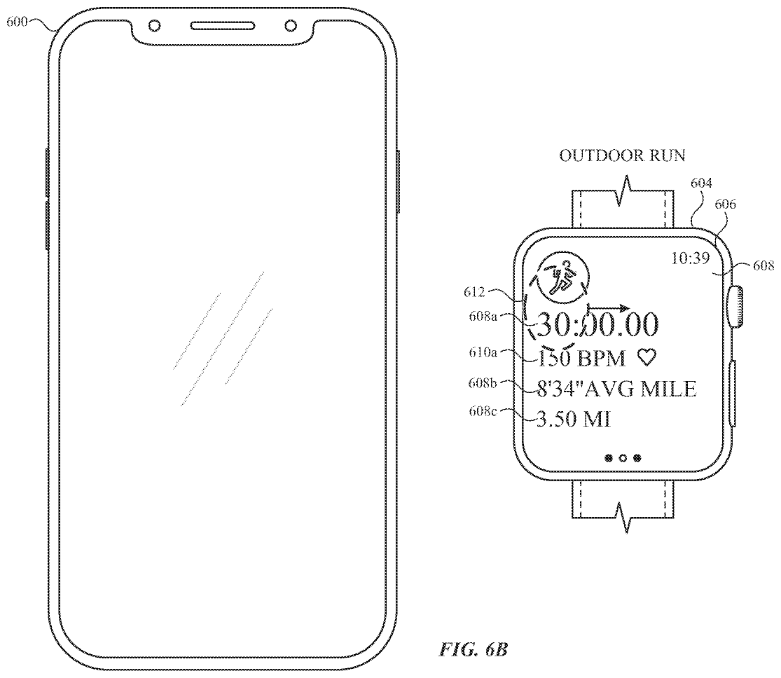

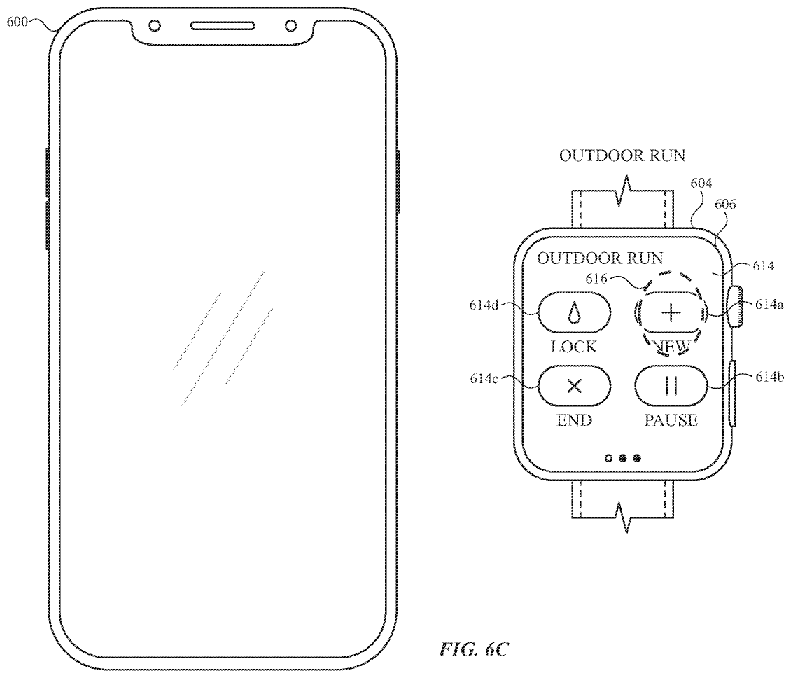

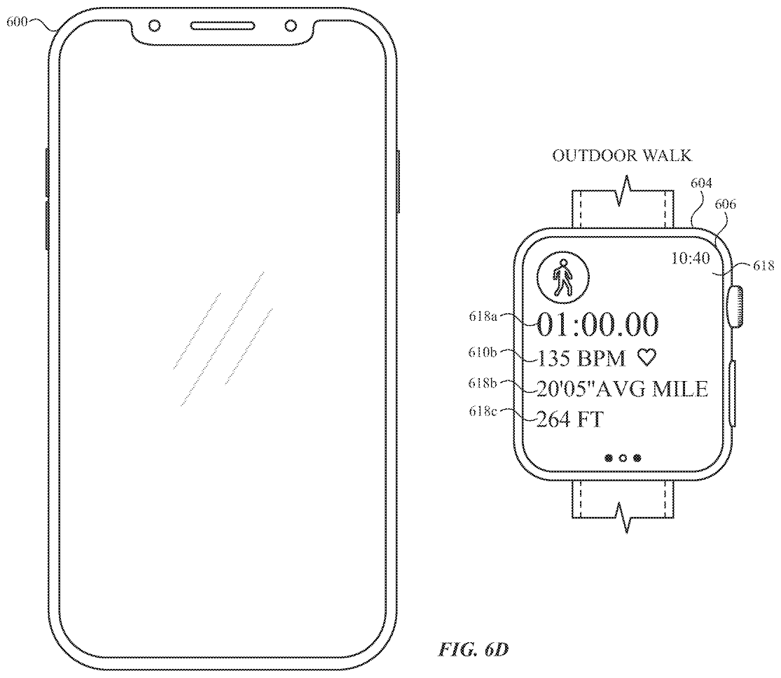

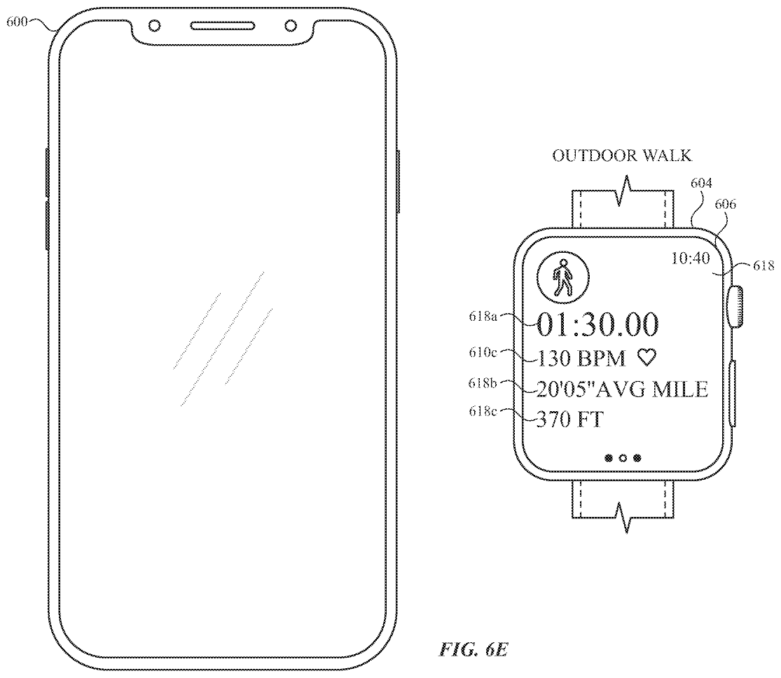

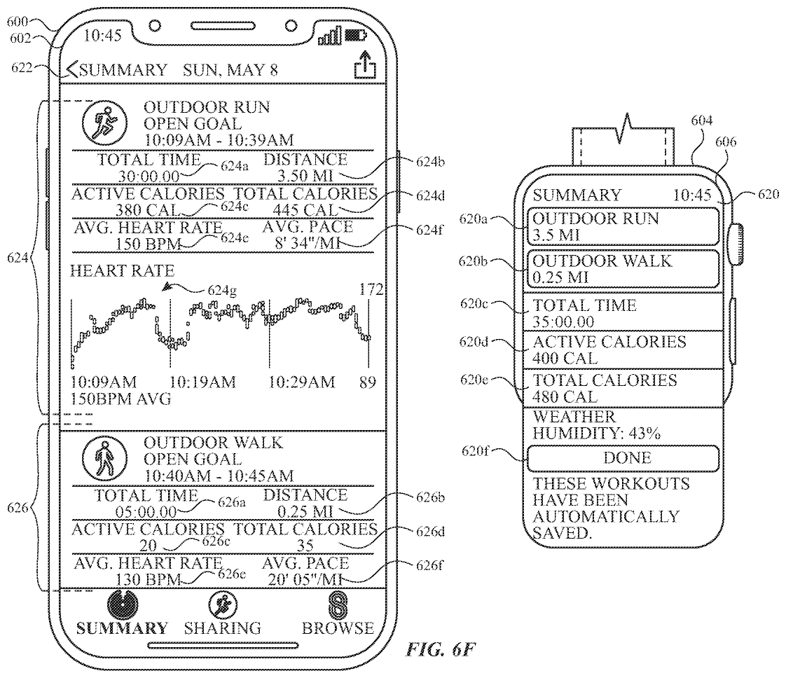

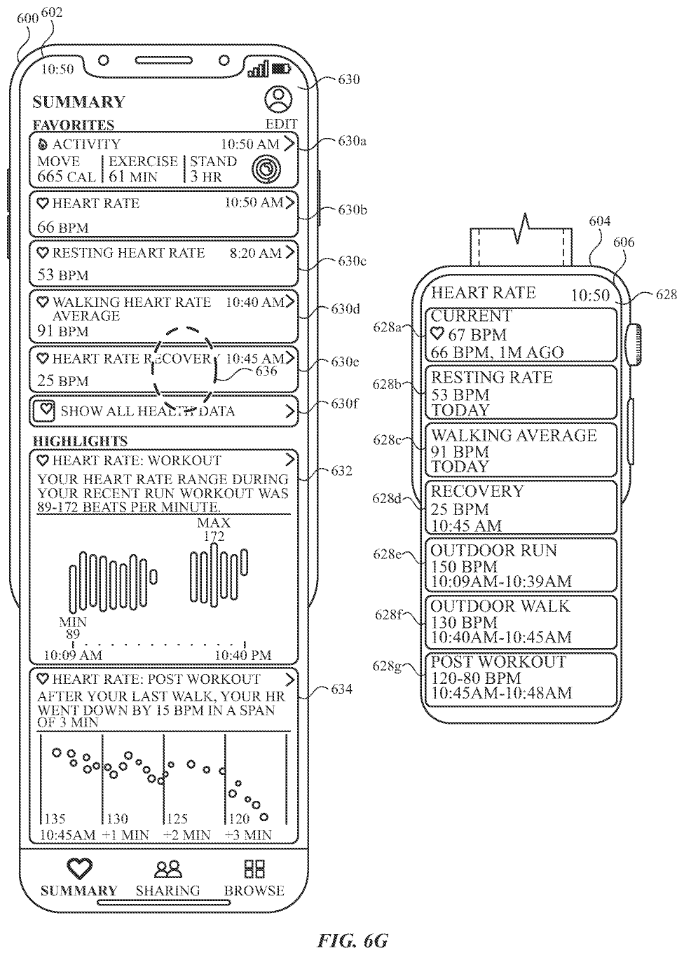

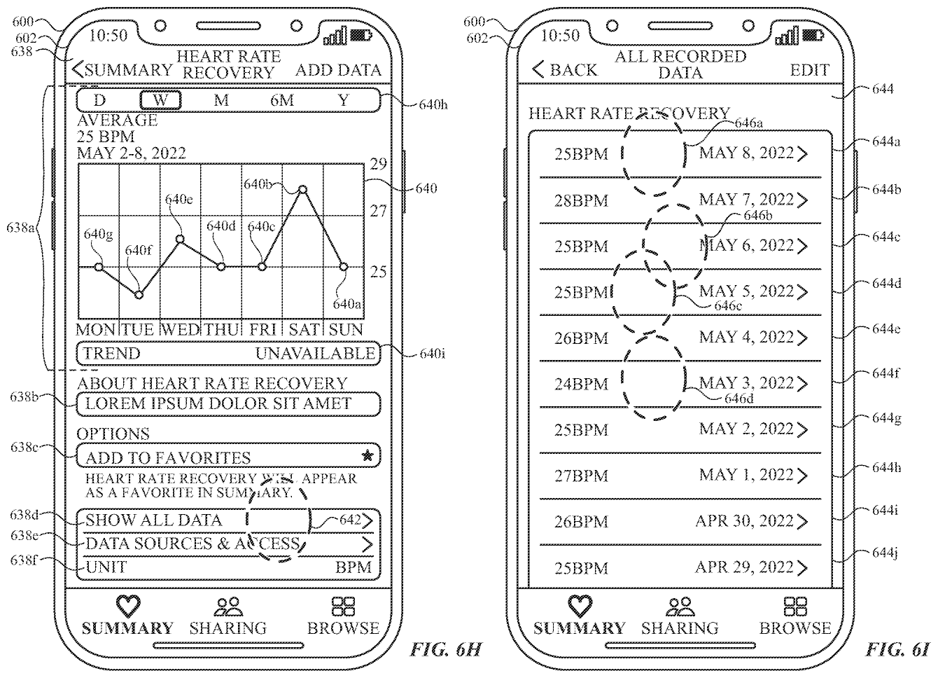

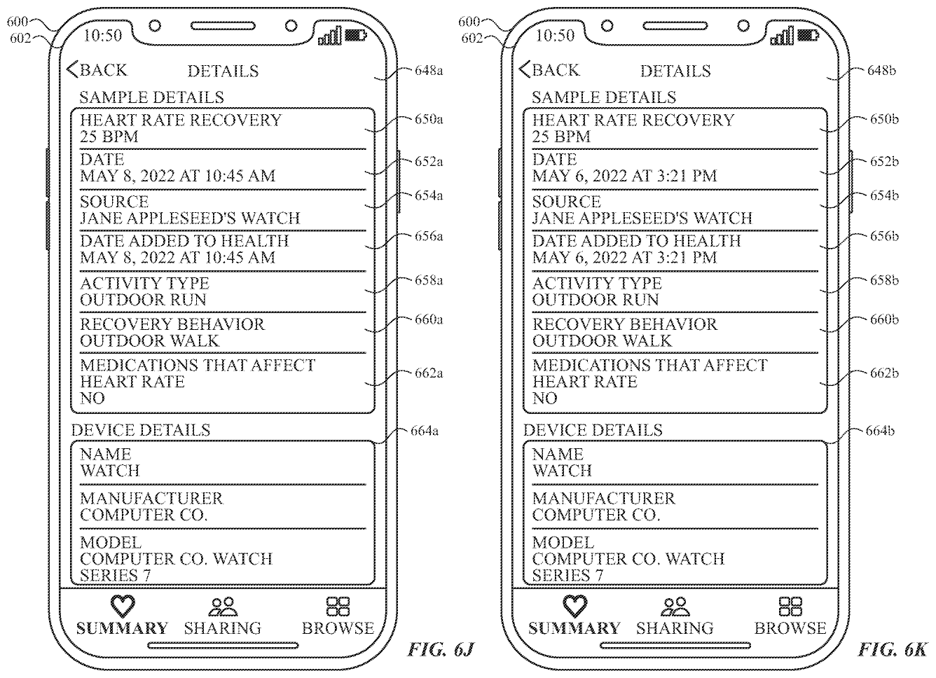

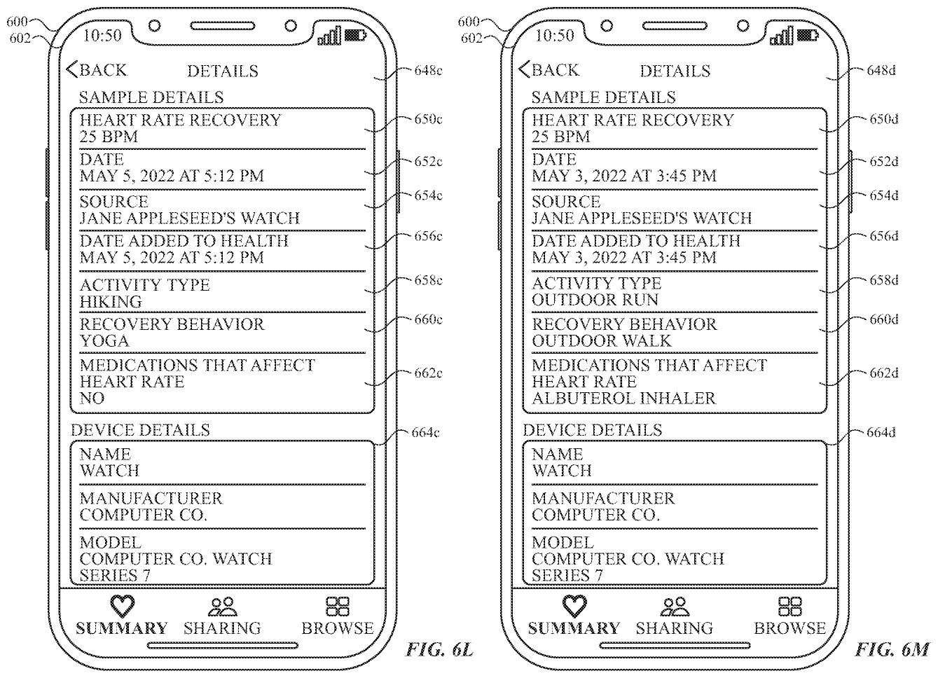

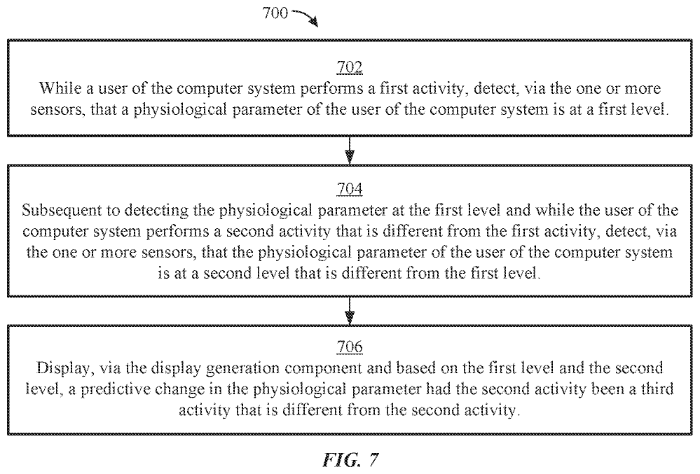

Below, FIGS. 1A-1, 2, 3, 4A-4B, and 5A-5B provide a description of exemplary devices for performing the techniques for managing event notifications. FIGS. 6A-6M illustrate exemplary user interfaces that include information related to physiological measurements. FIG. 7 is a flow diagram illustrating methods of displaying user interfaces including information related to physiological measurements, in accordance with some embodiments. The user interfaces in FIGS. 6A-6M are used to illustrate the processes described below, including the processes in FIG. 7.

The processes described below enhance the operability of the devices and make the user-device interfaces more efficient (e.g., by helping the user to provide proper inputs and reducing user mistakes when operating/interacting with the device) through various techniques, including by providing improved visual feedback to the user, reducing the number of inputs needed to perform an operation, providing additional control options without cluttering the user interface with additional displayed controls, performing an operation when a set of conditions has been met without requiring further user input, and/or additional techniques. These techniques also reduce power usage and improve battery life of the device by enabling the user to use the device more quickly and efficiently.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

Although the following description uses terms “first,” “second,” etc. to describe various elements, these elements should not be limited by the terms. In some embodiments, these terms are used to distinguish one element from another. For example, a first touch could be termed a second touch, and, similarly, a second touch could be termed a first touch, without departing from the scope of the various described embodiments. In some embodiments, the first touch and the second touch are two separate references to the same touch. In some embodiments, the first touch and the second touch are both touches, but they are not the same touch.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Embodiments of electronic devices, user interfaces for such devices, and associated processes for using such devices are described. In some embodiments, the device is a portable communications device, such as a mobile telephone, that also contains other functions, such as PDA and/or music player functions. Exemplary embodiments of portable multifunction devices include, without limitation, the iPhone®, iPod Touch®, and iPad® devices from Apple Inc. of Cupertino, California. Other portable electronic devices, such as laptops or tablet computers with touch-sensitive surfaces (e.g., touch screen displays and/or touchpads), are, optionally, used. It should also be understood that, in some embodiments, the device is not a portable communications device, but is a desktop computer with a touch-sensitive surface (e.g., a touch screen display and/or a touchpad). In some embodiments, the electronic device is a computer system that is in communication (e.g., via wireless communication, via wired communication) with a display generation component. The display generation component is configured to provide visual output, such as display via a CRT display, display via an LED display, or display via image projection. In some embodiments, the display generation component is integrated with the computer system. In some embodiments, the display generation component is separate from the computer system. As used herein, “displaying” content includes causing to display the content (e.g., video data rendered or decoded by display controller 156) by transmitting, via a wired or wireless connection, data (e.g., image data or video data) to an integrated or external display generation component to visually produce the content.

In the discussion that follows, an electronic device that includes a display and a touch-sensitive surface is described. It should be understood, however, that the electronic device optionally includes one or more other physical user-interface devices, such as a physical keyboard, a mouse, and/or a joystick.

The device typically supports a variety of applications, such as one or more of the following: a drawing application, a presentation application, a word processing application, a website creation application, a disk authoring application, a spreadsheet application, a gaming application, a telephone application, a video conferencing application, an e-mail application, an instant messaging application, a workout support application, a photo management application, a digital camera application, a digital video camera application, a web browsing application, a digital music player application, and/or a digital video player application.

The various applications that are executed on the device optionally use at least one common physical user-interface device, such as the touch-sensitive surface. One or more functions of the touch-sensitive surface as well as corresponding information displayed on the device are, optionally, adjusted and/or varied from one application to the next and/or within a respective application. In this way, a common physical architecture (such as the touch-sensitive surface) of the device optionally supports the variety of applications with user interfaces that are intuitive and transparent to the user.

Attention is now directed toward embodiments of portable devices with touch-sensitive displays. FIG. 1A is a block diagram illustrating portable multifunction device 100 with touch-sensitive display system 112 in accordance with some embodiments. Touch-sensitive display 112 is sometimes called a “touch screen” for convenience and is sometimes known as or called a “touch-sensitive display system.” Device 100 includes memory 102 (which optionally includes one or more computer-readable storage mediums), memory controller 122, one or more processing units (CPUs) 120, peripherals interface 118, RF circuitry 108, audio circuitry 110, speaker 111, microphone 113, input/output (I/O) subsystem 106, other input control devices 116, and external port 124. Device 100 optionally includes one or more optical sensors 164. Device 100 optionally includes one or more contact intensity sensors 165 for detecting intensity of contacts on device 100 (e.g., a touch-sensitive surface such as touch-sensitive display system 112 of device 100). Device 100 optionally includes one or more tactile output generators 167 for generating tactile outputs on device 100 (e.g., generating tactile outputs on a touch-sensitive surface such as touch-sensitive display system 112 of device 100 or touchpad 355 of device 300). These components optionally communicate over one or more communication buses or signal lines 103.

As used in the specification and claims, the term “intensity” of a contact on a touch-sensitive surface refers to the force or pressure (force per unit area) of a contact (e.g., a finger contact) on the touch-sensitive surface, or to a substitute (proxy) for the force or pressure of a contact on the touch-sensitive surface. The intensity of a contact has a range of values that includes at least four distinct values and more typically includes hundreds of distinct values (e.g., at least 256). Intensity of a contact is, optionally, determined (or measured) using various approaches and various sensors or combinations of sensors. For example, one or more force sensors underneath or adjacent to the touch-sensitive surface are, optionally, used to measure force at various points on the touch-sensitive surface. In some implementations, force measurements from multiple force sensors are combined (e.g., a weighted average) to determine an estimated force of a contact. Similarly, a pressure-sensitive tip of a stylus is, optionally, used to determine a pressure of the stylus on the touch-sensitive surface. Alternatively, the size of the contact area detected on the touch-sensitive surface and/or changes thereto, the capacitance of the touch-sensitive surface proximate to the contact and/or changes thereto, and/or the resistance of the touch-sensitive surface proximate to the contact and/or changes thereto are, optionally, used as a substitute for the force or pressure of the contact on the touch-sensitive surface. In some implementations, the substitute measurements for contact force or pressure are used directly to determine whether an intensity threshold has been exceeded (e.g., the intensity threshold is described in units corresponding to the substitute measurements). In some implementations, the substitute measurements for contact force or pressure are converted to an estimated force or pressure, and the estimated force or pressure is used to determine whether an intensity threshold has been exceeded (e.g., the intensity threshold is a pressure threshold measured in units of pressure). Using the intensity of a contact as an attribute of a user input allows for user access to additional device functionality that may otherwise not be accessible by the user on a reduced-size device with limited real estate for displaying affordances (e.g., on a touch-sensitive display) and/or receiving user input (e.g., via a touch-sensitive display, a touch-sensitive surface, or a physical/mechanical control such as a knob or a button).

As used in the specification and claims, the term “tactile output” refers to physical displacement of a device relative to a previous position of the device, physical displacement of a component (e.g., a touch-sensitive surface) of a device relative to another component (e.g., housing) of the device, or displacement of the component relative to a center of mass of the device that will be detected by a user with the user's sense of touch. For example, in situations where the device or the component of the device is in contact with a surface of a user that is sensitive to touch (e.g., a finger, palm, or other part of a user's hand), the tactile output generated by the physical displacement will be interpreted by the user as a tactile sensation corresponding to a perceived change in physical characteristics of the device or the component of the device. For example, movement of a touch-sensitive surface (e.g., a touch-sensitive display or trackpad) is, optionally, interpreted by the user as a “down click” or “up click” of a physical actuator button. In some cases, a user will feel a tactile sensation such as an “down click” or “up click” even when there is no movement of a physical actuator button associated with the touch-sensitive surface that is physically pressed (e.g., displaced) by the user's movements. As another example, movement of the touch-sensitive surface is, optionally, interpreted or sensed by the user as “roughness” of the touch-sensitive surface, even when there is no change in smoothness of the touch-sensitive surface. While such interpretations of touch by a user will be subject to the individualized sensory perceptions of the user, there are many sensory perceptions of touch that are common to a large majority of users. Thus, when a tactile output is described as corresponding to a particular sensory perception of a user (e.g., an “up click,” a “down click,” “roughness”), unless otherwise stated, the generated tactile output corresponds to physical displacement of the device or a component thereof that will generate the described sensory perception for a typical (or average) user.

It should be appreciated that device 100 is only one example of a portable multifunction device, and that device 100 optionally has more or fewer components than shown, optionally combines two or more components, or optionally has a different configuration or arrangement of the components. The various components shown in FIG. 1A are implemented in hardware, software, or a combination of both hardware and software, including one or more signal processing and/or application-specific integrated circuits.

Memory 102 optionally includes high-speed random access memory and optionally also includes non-volatile memory, such as one or more magnetic disk storage devices, flash memory devices, or other non-volatile solid-state memory devices. Memory controller 122 optionally controls access to memory 102 by other components of device 100.

Peripherals interface 118 can be used to couple input and output peripherals of the device to CPU 120 and memory 102. The one or more processors 120 run or execute various software programs (such as computer programs (e.g., including instructions)) and/or sets of instructions stored in memory 102 to perform various functions for device 100 and to process data. In some embodiments, peripherals interface 118, CPU 120, and memory controller 122 are, optionally, implemented on a single chip, such as chip 104. In some other embodiments, they are, optionally, implemented on separate chips.

RF (radio frequency) circuitry 108 receives and sends RF signals, also called electromagnetic signals. RF circuitry 108 converts electrical signals to/from electromagnetic signals and communicates with communications networks and other communications devices via the electromagnetic signals. RF circuitry 108 optionally includes well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth. RF circuitry 108 optionally communicates with networks, such as the Internet, also referred to as the World Wide Web (WWW), an intranet and/or a wireless network, such as a cellular telephone network, a wireless local area network (LAN) and/or a metropolitan area network (MAN), and other devices by wireless communication. The RF circuitry 108 optionally includes well-known circuitry for detecting near field communication (NFC) fields, such as by a short-range communication radio. The wireless communication optionally uses any of a plurality of communications standards, protocols, and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Bluetooth Low Energy (BTLE), Wireless Fidelity (Wi-Fi) (e.g., IEEE 802.11a, IEEE 802.11b, IEEE 802.11g, IEEE 802.11n, and/or IEEE 802.11 ac), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

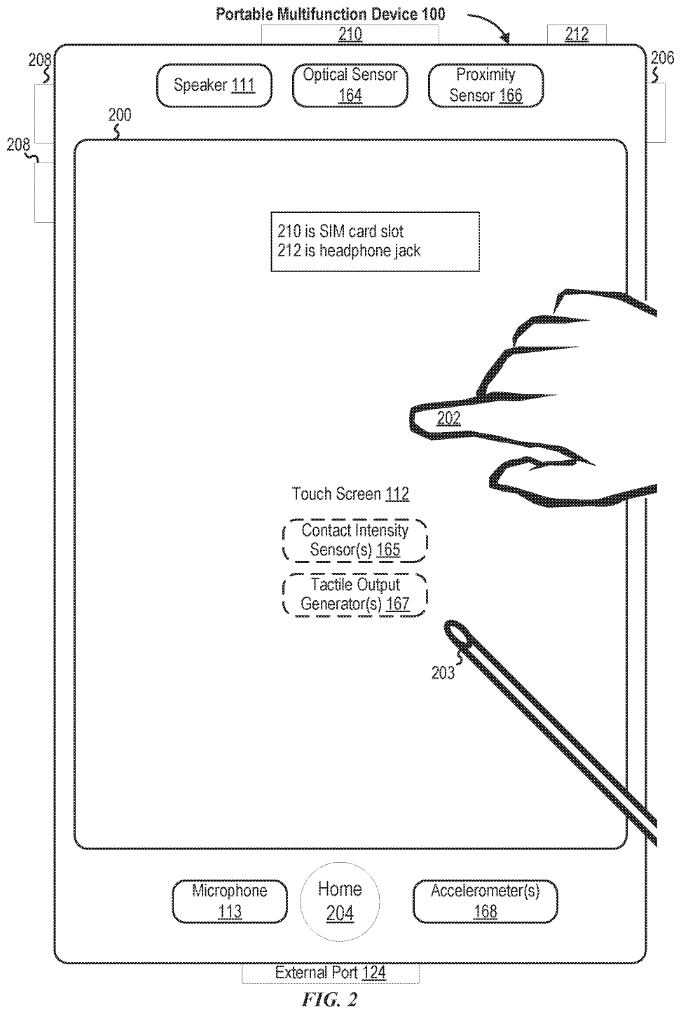

Audio circuitry 110, speaker 111, and microphone 113 provide an audio interface between a user and device 100. Audio circuitry 110 receives audio data from peripherals interface 118, converts the audio data to an electrical signal, and transmits the electrical signal to speaker 111. Speaker 111 converts the electrical signal to human-audible sound waves. Audio circuitry 110 also receives electrical signals converted by microphone 113 from sound waves. Audio circuitry 110 converts the electrical signal to audio data and transmits the audio data to peripherals interface 118 for processing. Audio data is, optionally, retrieved from and/or transmitted to memory 102 and/or RF circuitry 108 by peripherals interface 118. In some embodiments, audio circuitry 110 also includes a headset jack (e.g., 212, FIG. 2). The headset jack provides an interface between audio circuitry 110 and removable audio input/output peripherals, such as output-only headphones or a headset with both output (e.g., a headphone for one or both ears) and input (e.g., a microphone).

I/O subsystem 106 couples input/output peripherals on device 100, such as touch screen 112 and other input control devices 116, to peripherals interface 118. I/O subsystem 106 optionally includes display controller 156, optical sensor controller 158, depth camera controller 169, intensity sensor controller 159, haptic feedback controller 161, and one or more input controllers 160 for other input or control devices. The one or more input controllers 160 receive/send electrical signals from/to other input control devices 116. The other input control devices 116 optionally include physical buttons (e.g., push buttons, rocker buttons, etc.), dials, slider switches, joysticks, click wheels, and so forth. In some embodiments, input controller(s) 160 are, optionally, coupled to any (or none) of the following: a keyboard, an infrared port, a USB port, and a pointer device such as a mouse. The one or more buttons (e.g., 208, FIG. 2) optionally include an up/down button for volume control of speaker 111 and/or microphone 113. The one or more buttons optionally include a push button (e.g., 206, FIG. 2). In some embodiments, the electronic device is a computer system that is in communication (e.g., via wireless communication, via wired communication) with one or more input devices. In some embodiments, the one or more input devices include a touch-sensitive surface (e.g., a trackpad, as part of a touch-sensitive display). In some embodiments, the one or more input devices include one or more camera sensors (e.g., one or more optical sensors 164 and/or one or more depth camera sensors 175), such as for tracking a user's gestures (e.g., hand gestures and/or air gestures) as input. In some embodiments, the one or more input devices are integrated with the computer system. In some embodiments, the one or more input devices are separate from the computer system. In some embodiments, an air gesture is a gesture that is detected without the user touching an input element that is part of the device (or independently of an input element that is a part of the device) and is based on detected motion of a portion of the user's body through the air including motion of the user's body relative to an absolute reference (e.g., an angle of the user's arm relative to the ground or a distance of the user's hand relative to the ground), relative to another portion of the user's body (e.g., movement of a hand of the user relative to a shoulder of the user, movement of one hand of the user relative to another hand of the user, and/or movement of a finger of the user relative to another finger or portion of a hand of the user), and/or absolute motion of a portion of the user's body (e.g., a tap gesture that includes movement of a hand in a predetermined pose by a predetermined amount and/or speed, or a shake gesture that includes a predetermined speed or amount of rotation of a portion of the user's body).

A quick press of the push button optionally disengages a lock of touch screen 112 or optionally begins a process that uses gestures on the touch screen to unlock the device, as described in U.S. patent application Ser. No. 11/322,549, “Unlocking a Device by Performing Gestures on an Unlock Image,” filed Dec. 23, 2005, U.S. Pat. No. 7,657,849, which is hereby incorporated by reference in its entirety. A longer press of the push button (e.g., 206) optionally turns power to device 100 on or off. The functionality of one or more of the buttons are, optionally, user-customizable. Touch screen 112 is used to implement virtual or soft buttons and one or more soft keyboards.

Touch-sensitive display 112 provides an input interface and an output interface between the device and a user. Display controller 156 receives and/or sends electrical signals from/to touch screen 112. Touch screen 112 displays visual output to the user. The visual output optionally includes graphics, text, icons, video, and any combination thereof (collectively termed “graphics”). In some embodiments, some or all of the visual output optionally corresponds to user-interface objects.

Touch screen 112 has a touch-sensitive surface, sensor, or set of sensors that accepts input from the user based on haptic and/or tactile contact. Touch screen 112 and display controller 156 (along with any associated modules and/or sets of instructions in memory 102) detect contact (and any movement or breaking of the contact) on touch screen 112 and convert the detected contact into interaction with user-interface objects (e.g., one or more soft keys, icons, web pages, or images) that are displayed on touch screen 112. In an exemplary embodiment, a point of contact between touch screen 112 and the user corresponds to a finger of the user.

Touch screen 112 optionally uses LCD (liquid crystal display) technology, LPD (light emitting polymer display) technology, or LED (light emitting diode) technology, although other display technologies are used in other embodiments. Touch screen 112 and display controller 156 optionally detect contact and any movement or breaking thereof using any of a plurality of touch sensing technologies now known or later developed, including but not limited to capacitive, resistive, infrared, and surface acoustic wave technologies, as well as other proximity sensor arrays or other elements for determining one or more points of contact with touch screen 112. In an exemplary embodiment, projected mutual capacitance sensing technology is used, such as that found in the iPhone® and iPod Touch® from Apple Inc. of Cupertino, California.

A touch-sensitive display in some embodiments of touch screen 112 is, optionally, analogous to the multi-touch sensitive touchpads described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1, each of which is hereby incorporated by reference in its entirety. However, touch screen 112 displays visual output from device 100, whereas touch-sensitive touchpads do not provide visual output.

A touch-sensitive display in some embodiments of touch screen 112 is described in the following applications: (1) U.S. patent application Ser. No. 11/381,313, “Multipoint Touch Surface Controller,” filed May 2, 2006; (2) U.S. patent application Ser. No. 10/840,862, “Multipoint Touchscreen,” filed May 6, 2004; (3) U.S. patent application Ser. No. 10/903,964, “Gestures For Touch Sensitive Input Devices,” filed Jul. 30, 2004; (4) U.S. patent application Ser. No. 11/048,264, “Gestures For Touch Sensitive Input Devices,” filed Jan. 31, 2005; (5) U.S. patent application Ser. No. 11/038,590, “Mode-Based Graphical User Interfaces For Touch Sensitive Input Devices,” filed Jan. 18, 2005; (6) U.S. patent application Ser. No. 11/228,758, “Virtual Input Device Placement On A Touch Screen User Interface,” filed Sep. 16, 2005; (7) U.S. patent application Ser. No. 11/228,700, “Operation Of A Computer With A Touch Screen Interface,” filed Sep. 16, 2005; (8) U.S. patent application Ser. No. 11/228,737, “Activating Virtual Keys Of A Touch-Screen Virtual Keyboard,” filed Sep. 16, 2005; and (9) U.S. patent application Ser. No. 11/367,749, “Multi-Functional Hand-Held Device,” filed Mar. 3, 2006. All of these applications are incorporated by reference herein in their entirety.

Touch screen 112 optionally has a video resolution in excess of 100 dpi. In some embodiments, the touch screen has a video resolution of approximately 160 dpi. The user optionally makes contact with touch screen 112 using any suitable object or appendage, such as a stylus, a finger, and so forth. In some embodiments, the user interface is designed to work primarily with finger-based contacts and gestures, which can be less precise than stylus-based input due to the larger area of contact of a finger on the touch screen. In some embodiments, the device translates the rough finger-based input into a precise pointer/cursor position or command for performing the actions desired by the user.

In some embodiments, in addition to the touch screen, device 100 optionally includes a touchpad for activating or deactivating particular functions. In some embodiments, the touchpad is a touch-sensitive area of the device that, unlike the touch screen, does not display visual output. The touchpad is, optionally, a touch-sensitive surface that is separate from touch screen 112 or an extension of the touch-sensitive surface formed by the touch screen.

Device 100 also includes power system 162 for powering the various components. Power system 162 optionally includes a power management system, one or more power sources (e.g., battery, alternating current (AC)), a recharging system, a power failure detection circuit, a power converter or inverter, a power status indicator (e.g., a light-emitting diode (LED)) and any other components associated with the generation, management and distribution of power in portable devices.

Device 100 optionally also includes one or more optical sensors 164. FIG. 1A shows an optical sensor coupled to optical sensor controller 158 in I/O subsystem 106. Optical sensor 164 optionally includes charge-coupled device (CCD) or complementary metal-oxide semiconductor (CMOS) phototransistors. Optical sensor 164 receives light from the environment, projected through one or more lenses, and converts the light to data representing an image. In conjunction with imaging module 143 (also called a camera module), optical sensor 164 optionally captures still images or video. In some embodiments, an optical sensor is located on the back of device 100, opposite touch screen display 112 on the front of the device so that the touch screen display is enabled for use as a viewfinder for still and/or video image acquisition. In some embodiments, an optical sensor is located on the front of the device so that the user's image is, optionally, obtained for video conferencing while the user views the other video conference participants on the touch screen display. In some embodiments, the position of optical sensor 164 can be changed by the user (e.g., by rotating the lens and the sensor in the device housing) so that a single optical sensor 164 is used along with the touch screen display for both video conferencing and still and/or video image acquisition.

Device 100 optionally also includes one or more depth camera sensors 175. FIG. 1A shows a depth camera sensor coupled to depth camera controller 169 in I/O subsystem 106. Depth camera sensor 175 receives data from the environment to create a three dimensional model of an object (e.g., a face) within a scene from a viewpoint (e.g., a depth camera sensor). In some embodiments, in conjunction with imaging module 143 (also called a camera module), depth camera sensor 175 is optionally used to determine a depth map of different portions of an image captured by the imaging module 143. In some embodiments, a depth camera sensor is located on the front of device 100 so that the user's image with depth information is, optionally, obtained for video conferencing while the user views the other video conference participants on the touch screen display and to capture selfies with depth map data. In some embodiments, the depth camera sensor 175 is located on the back of device, or on the back and the front of the device 100. In some embodiments, the position of depth camera sensor 175 can be changed by the user (e.g., by rotating the lens and the sensor in the device housing) so that a depth camera sensor 175 is used along with the touch screen display for both video conferencing and still and/or video image acquisition.

Device 100 optionally also includes one or more contact intensity sensors 165. FIG. 1A shows a contact intensity sensor coupled to intensity sensor controller 159 in I/O subsystem 106. Contact intensity sensor 165 optionally includes one or more piezoresistive strain gauges, capacitive force sensors, electric force sensors, piezoelectric force sensors, optical force sensors, capacitive touch-sensitive surfaces, or other intensity sensors (e.g., sensors used to measure the force (or pressure) of a contact on a touch-sensitive surface). Contact intensity sensor 165 receives contact intensity information (e.g., pressure information or a proxy for pressure information) from the environment. In some embodiments, at least one contact intensity sensor is collocated with, or proximate to, a touch-sensitive surface (e.g., touch-sensitive display system 112). In some embodiments, at least one contact intensity sensor is located on the back of device 100, opposite touch screen display 112, which is located on the front of device 100.

Device 100 optionally also includes one or more proximity sensors 166. FIG. 1A shows proximity sensor 166 coupled to peripherals interface 118. Alternately, proximity sensor 166 is, optionally, coupled to input controller 160 in I/O subsystem 106. Proximity sensor 166 optionally performs as described in U.S. patent application Ser. No. 11/241,839, “Proximity Detector In Handheld Device”; Ser. No. 11/240,788, “Proximity Detector In Handheld Device”; Ser. No. 11/620,702, “Using Ambient Light Sensor To Augment Proximity Sensor Output”; Ser. No. 11/586,862, “Automated Response To And Sensing Of User Activity In Portable Devices”; and Ser. No. 11/638,251, “Methods And Systems For Automatic Configuration Of Peripherals,” which are hereby incorporated by reference in their entirety. In some embodiments, the proximity sensor turns off and disables touch screen 112 when the multifunction device is placed near the user's ear (e.g., when the user is making a phone call).

Device 100 optionally also includes one or more tactile output generators 167. FIG. 1A shows a tactile output generator coupled to haptic feedback controller 161 in I/O subsystem 106. Tactile output generator 167 optionally includes one or more electroacoustic devices such as speakers or other audio components and/or electromechanical devices that convert energy into linear motion such as a motor, solenoid, electroactive polymer, piezoelectric actuator, electrostatic actuator, or other tactile output generating component (e.g., a component that converts electrical signals into tactile outputs on the device). Contact intensity sensor 165 receives tactile feedback generation instructions from haptic feedback module 133 and generates tactile outputs on device 100 that are capable of being sensed by a user of device 100. In some embodiments, at least one tactile output generator is collocated with, or proximate to, a touch-sensitive surface (e.g., touch-sensitive display system 112) and, optionally, generates a tactile output by moving the touch-sensitive surface vertically (e.g., in/out of a surface of device 100) or laterally (e.g., back and forth in the same plane as a surface of device 100). In some embodiments, at least one tactile output generator sensor is located on the back of device 100, opposite touch screen display 112, which is located on the front of device 100.

Device 100 optionally also includes one or more accelerometers 168. FIG. 1A shows accelerometer 168 coupled to peripherals interface 118. Alternately, accelerometer 168 is, optionally, coupled to an input controller 160 in I/O subsystem 106. Accelerometer 168 optionally performs as described in U.S. Patent Publication No. 20050190059, “Acceleration-based Theft Detection System for Portable Electronic Devices,” and U.S. Patent Publication No. 20060017692, “Methods And Apparatuses For Operating A Portable Device Based On An Accelerometer,” both of which are incorporated by reference herein in their entirety. In some embodiments, information is displayed on the touch screen display in a portrait view or a landscape view based on an analysis of data received from the one or more accelerometers. Device 100 optionally includes, in addition to accelerometer(s) 168, a magnetometer and a GPS (or GLONASS or other global navigation system) receiver for obtaining information concerning the location and orientation (e.g., portrait or landscape) of device 100.

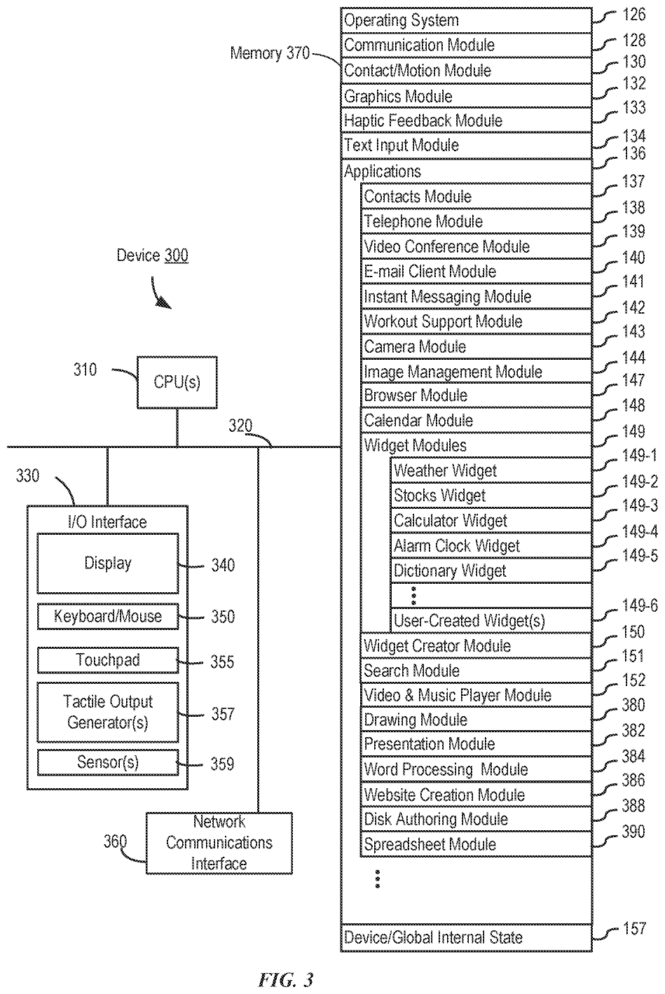

In some embodiments, the software components stored in memory 102 include operating system 126, communication module (or set of instructions) 128, contact/motion module (or set of instructions) 130, graphics module (or set of instructions) 132, text input module (or set of instructions) 134, Global Positioning System (GPS) module (or set of instructions) 135, and applications (or sets of instructions) 136. Furthermore, in some embodiments, memory 102 (FIG. 1A) or 370 (FIG. 3) stores device/global internal state 157, as shown in FIGS. 1A and 3. Device/global internal state 157 includes one or more of: active application state, indicating which applications, if any, are currently active; display state, indicating what applications, views or other information occupy various regions of touch screen display 112; sensor state, including information obtained from the device's various sensors and input control devices 116; and location information concerning the device's location and/or attitude.

Operating system 126 (e.g., Darwin, RTXC, LINUX, UNIX, OS X, iOS, WINDOWS, or an embedded operating system such as VxWorks) includes various software components and/or drivers for controlling and managing general system tasks (e.g., memory management, storage device control, power management, etc.) and facilitates communication between various hardware and software components.

Communication module 128 facilitates communication with other devices over one or more external ports 124 and also includes various software components for handling data received by RF circuitry 108 and/or external port 124. External port 124 (e.g., Universal Serial Bus (USB), FIREWIRE, etc.) is adapted for coupling directly to other devices or indirectly over a network (e.g., the Internet, wireless LAN, etc.). In some embodiments, the external port is a multi-pin (e.g., 30-pin) connector that is the same as, or similar to and/or compatible with, the 30-pin connector used on iPod® (trademark of Apple Inc.) devices.

Contact/motion module 130 optionally detects contact with touch screen 112 (in conjunction with display controller 156) and other touch-sensitive devices (e.g., a touchpad or physical click wheel). Contact/motion module 130 includes various software components for performing various operations related to detection of contact, such as determining if contact has occurred (e.g., detecting a finger-down event), determining an intensity of the contact (e.g., the force or pressure of the contact or a substitute for the force or pressure of the contact), determining if there is movement of the contact and tracking the movement across the touch-sensitive surface (e.g., detecting one or more finger-dragging events), and determining if the contact has ceased (e.g., detecting a finger-up event or a break in contact). Contact/motion module 130 receives contact data from the touch-sensitive surface. Determining movement of the point of contact, which is represented by a series of contact data, optionally includes determining speed (magnitude), velocity (magnitude and direction), and/or an acceleration (a change in magnitude and/or direction) of the point of contact. These operations are, optionally, applied to single contacts (e.g., one finger contacts) or to multiple simultaneous contacts (e.g., “multitouch”/multiple finger contacts). In some embodiments, contact/motion module 130 and display controller 156 detect contact on a touchpad.

In some embodiments, contact/motion module 130 uses a set of one or more intensity thresholds to determine whether an operation has been performed by a user (e.g., to determine whether a user has “clicked” on an icon). In some embodiments, at least a subset of the intensity thresholds are determined in accordance with software parameters (e.g., the intensity thresholds are not determined by the activation thresholds of particular physical actuators and can be adjusted without changing the physical hardware of device 100). For example, a mouse “click” threshold of a trackpad or touch screen display can be set to any of a large range of predefined threshold values without changing the trackpad or touch screen display hardware. Additionally, in some implementations, a user of the device is provided with software settings for adjusting one or more of the set of intensity thresholds (e.g., by adjusting individual intensity thresholds and/or by adjusting a plurality of intensity thresholds at once with a system-level click “intensity” parameter).

Contact/motion module 130 optionally detects a gesture input by a user. Different gestures on the touch-sensitive surface have different contact patterns (e.g., different motions, timings, and/or intensities of detected contacts). Thus, a gesture is, optionally, detected by detecting a particular contact pattern. For example, detecting a finger tap gesture includes detecting a finger-down event followed by detecting a finger-up (liftoff) event at the same position (or substantially the same position) as the finger-down event (e.g., at the position of an icon). As another example, detecting a finger swipe gesture on the touch-sensitive surface includes detecting a finger-down event followed by detecting one or more finger-dragging events, and subsequently followed by detecting a finger-up (liftoff) event.

Graphics module 132 includes various known software components for rendering and displaying graphics on touch screen 112 or other display, including components for changing the visual impact (e.g., brightness, transparency, saturation, contrast, or other visual property) of graphics that are displayed. As used herein, the term “graphics” includes any object that can be displayed to a user, including, without limitation, text, web pages, icons (such as user-interface objects including soft keys), digital images, videos, animations, and the like.

In some embodiments, graphics module 132 stores data representing graphics to be used. Each graphic is, optionally, assigned a corresponding code. Graphics module 132 receives, from applications etc., one or more codes specifying graphics to be displayed along with, if necessary, coordinate data and other graphic property data, and then generates screen image data to output to display controller 156.

Haptic feedback module 133 includes various software components for generating instructions used by tactile output generator(s) 167 to produce tactile outputs at one or more locations on device 100 in response to user interactions with device 100.

Text input module 134, which is, optionally, a component of graphics module 132, provides soft keyboards for entering text in various applications (e.g., contacts 137, e-mail 140, IM 141, browser 147, and any other application that needs text input).

GPS module 135 determines the location of the device and provides this information for use in various applications (e.g., to telephone 138 for use in location-based dialing; to camera 143 as picture/video metadata; and to applications that provide location-based services such as weather widgets, local yellow page widgets, and map/navigation widgets).

Applications 136 optionally include the following modules (or sets of instructions), or a subset or superset thereof:
Contacts module 137 (sometimes called an address book or contact list);
Telephone module 138;
Video conference module 139;
E-mail client module 140;
Instant messaging (IM) module 141;
Workout support module 142;
Camera module 143 for still and/or video images;
Image management module 144;
Video player module;
Music player module;
Browser module 147;
Calendar module 148;
Widget modules 149, which optionally include one or more of: weather widget 149-1, stocks widget 149-2, calculator widget 149-3, alarm clock widget 149-4, dictionary widget 149-5, and other widgets obtained by the user, as well as user-created widgets 149-6;
Widget creator module 150 for making user-created widgets 149-6;
Search module 151;
Video and music player module 152, which merges video player module and music player module;
Notes module 153;
Map module 154; and/or
Online video module 155.

Examples of other applications 136 that are, optionally, stored in memory 102 include other word processing applications, other image editing applications, drawing applications, presentation applications, JAVA-enabled applications, encryption, digital rights management, voice recognition, and voice replication.

In conjunction with touch screen 112, display controller 156, contact/motion module 130, graphics module 132, and text input module 134, contacts module 137 are, optionally, used to manage an address book or contact list (e.g., stored in application internal state 192 of contacts module 137 in memory 102 or memory 370), including: adding name(s) to the address book; deleting name(s) from the address book; associating telephone number(s), e-mail address(es), physical address(es) or other information with a name; associating an image with a name; categorizing and sorting names; providing telephone numbers or e-mail addresses to initiate and/or facilitate communications by telephone 138, video conference module 139, e-mail 140, or IM 141; and so forth.

In conjunction with RF circuitry 108, audio circuitry 110, speaker 111, microphone 113, touch screen 112, display controller 156, contact/motion module 130, graphics module 132, and text input module 134, telephone module 138 are optionally, used to enter a sequence of characters corresponding to a telephone number, access one or more telephone numbers in contacts module 137, modify a telephone number that has been entered, dial a respective telephone number, conduct a conversation, and disconnect or hang up when the conversation is completed. As noted above, the wireless communication optionally uses any of a plurality of communications standards, protocols, and technologies.

In conjunction with RF circuitry 108, audio circuitry 110, speaker 111, microphone 113, touch screen 112, display controller 156, optical sensor 164, optical sensor controller 158, contact/motion module 130, graphics module 132, text input module 134, contacts module 137, and telephone module 138, video conference module 139 includes executable instructions to initiate, conduct, and terminate a video conference between a user and one or more other participants in accordance with user instructions.

In conjunction with RF circuitry 108, touch screen 112, display controller 156, contact/motion module 130, graphics module 132, and text input module 134, e-mail client module 140 includes executable instructions to create, send, receive, and manage e-mail in response to user instructions. In conjunction with image management module 144, e-mail client module 140 makes it very easy to create and send e-mails with still or video images taken with camera module 143.

In conjunction with RF circuitry 108, touch screen 112, display controller 156, contact/motion module 130, graphics module 132, and text input module 134, the instant messaging module 141 includes executable instructions to enter a sequence of characters corresponding to an instant message, to modify previously entered characters, to transmit a respective instant message (for example, using a Short Message Service (SMS) or Multimedia Message Service (MMS) protocol for telephony-based instant messages or using XMPP, SIMPLE, or IMPS for Internet-based instant messages), to receive instant messages, and to view received instant messages. In some embodiments, transmitted and/or received instant messages optionally include graphics, photos, audio files, video files and/or other attachments as are supported in an MMS and/or an Enhanced Messaging Service (EMS). As used herein, “instant messaging” refers to both telephony-based messages (e.g., messages sent using SMS or MMS) and Internet-based messages (e.g., messages sent using XMPP, SIMPLE, or IMPS).

In conjunction with RF circuitry 108, touch screen 112, display controller 156, contact/motion module 130, graphics module 132, text input module 134, GPS module 135, map module 154, and music player module, workout support module 142 includes executable instructions to create workouts (e.g., with time, distance, and/or calorie burning goals); communicate with workout sensors (sports devices); receive workout sensor data; calibrate sensors used to monitor a workout; select and play music for a workout; and display, store, and transmit workout data.

In conjunction with touch screen 112, display controller 156, optical sensor(s) 164, optical sensor controller 158, contact/motion module 130, graphics module 132, and image management module 144, camera module 143 includes executable instructions to capture still images or video (including a video stream) and store them into memory 102, modify characteristics of a still image or video, or delete a still image or video from memory 102.

In conjunction with touch screen 112, display controller 156, contact/motion module 130, graphics module 132, text input module 134, and camera module 143, image management module 144 includes executable instructions to arrange, modify (e.g., edit), or otherwise manipulate, label, delete, present (e.g., in a digital slide show or album), and store still and/or video images.

In conjunction with RF circuitry 108, touch screen 112, display controller 156, contact/motion module 130, graphics module 132, and text input module 134, browser module 147 includes executable instructions to browse the Internet in accordance with user instructions, including searching, linking to, receiving, and displaying web pages or portions thereof, as well as attachments and other files linked to web pages.

In conjunction with RF circuitry 108, touch screen 112, display controller 156, contact/motion module 130, graphics module 132, text input module 134, e-mail client module 140, and browser module 147, calendar module 148 includes executable instructions to create, display, modify, and store calendars and data associated with calendars (e.g., calendar entries, to-do lists, etc.) in accordance with user instructions.

In conjunction with RF circuitry 108, touch screen 112, display controller 156, contact/motion module 130, graphics module 132, text input module 134, and browser module 147, widget modules 149 are mini-applications that are, optionally, downloaded and used by a user (e.g., weather widget 149-1, stocks widget 149-2, calculator widget 149-3, alarm clock widget 149-4, and dictionary widget 149-5) or created by the user (e.g., user-created widget 149-6). In some embodiments, a widget includes an HTML (Hypertext Markup Language) file, a CSS (Cascading Style Sheets) file, and a JavaScript file. In some embodiments, a widget includes an XML (Extensible Markup Language) file and a JavaScript file (e.g., Yahoo!Widgets).

In conjunction with RF circuitry 108, touch screen 112, display controller 156, contact/motion module 130, graphics module 132, text input module 134, and browser module 147, the widget creator module 150 are, optionally, used by a user to create widgets (e.g., turning a user-specified portion of a web page into a widget).

In conjunction with touch screen 112, display controller 156, contact/motion module 130, graphics module 132, and text input module 134, search module 151 includes executable instructions to search for text, music, sound, image, video, and/or other files in memory 102 that match one or more search criteria (e.g., one or more user-specified search terms) in accordance with user instructions.

In conjunction with touch screen 112, display controller 156, contact/motion module 130, graphics module 132, audio circuitry 110, speaker 111, RF circuitry 108, and browser module 147, video and music player module 152 includes executable instructions that allow the user to download and play back recorded music and other sound files stored in one or more file formats, such as MP3 or AAC files, and executable instructions to display, present, or otherwise play back videos (e.g., on touch screen 112 or on an external, connected display via external port 124). In some embodiments, device 100 optionally includes the functionality of an MP3 player, such as an iPod (trademark of Apple Inc.).

In conjunction with touch screen 112, display controller 156, contact/motion module 130, graphics module 132, and text input module 134, notes module 153 includes executable instructions to create and manage notes, to-do lists, and the like in accordance with user instructions.

In conjunction with RF circuitry 108, touch screen 112, display controller 156, contact/motion module 130, graphics module 132, text input module 134, GPS module 135, and browser module 147, map module 154 are, optionally, used to receive, display, modify, and store maps and data associated with maps (e.g., driving directions, data on stores and other points of interest at or near a particular location, and other location-based data) in accordance with user instructions.

In conjunction with touch screen 112, display controller 156, contact/motion module 130, graphics module 132, audio circuitry 110, speaker 111, RF circuitry 108, text input module 134, e-mail client module 140, and browser module 147, online video module 155 includes instructions that allow the user to access, browse, receive (e.g., by streaming and/or download), play back (e.g., on the touch screen or on an external, connected display via external port 124), send an e-mail with a link to a particular online video, and otherwise manage online videos in one or more file formats, such as H.264. In some embodiments, instant messaging module 141, rather than e-mail client module 140, is used to send a link to a particular online video. Additional description of the online video application can be found in U.S. Provisional Patent Application No. 60/936,562, “Portable Multifunction Device, Method, and Graphical User Interface for Playing Online Videos,” filed Jun. 20, 2007, and U.S. patent application Ser. No. 11/968,067, “Portable Multifunction Device, Method, and Graphical User Interface for Playing Online Videos,” filed Dec. 31, 2007, the contents of which are hereby incorporated by reference in their entirety.

Each of the above-identified modules and applications corresponds to a set of executable instructions for performing one or more functions described above and the methods described in this application (e.g., the computer-implemented methods and other information processing methods described herein). These modules (e.g., sets of instructions) need not be implemented as separate software programs (such as computer programs (e.g., including instructions)), procedures, or modules, and thus various subsets of these modules are, optionally, combined or otherwise rearranged in various embodiments. For example, video player module is, optionally, combined with music player module into a single module (e.g., video and music player module 152, FIG. 1A). In some embodiments, memory 102 optionally stores a subset of the modules and data structures identified above. Furthermore, memory 102 optionally stores additional modules and data structures not described above.

In some embodiments, device 100 is a device where operation of a predefined set of functions on the device is performed exclusively through a touch screen and/or a touchpad. By using a touch screen and/or a touchpad as the primary input control device for operation of device 100, the number of physical input control devices (such as push buttons, dials, and the like) on device 100 is, optionally, reduced.

The predefined set of functions that are performed exclusively through a touch screen and/or a touchpad optionally include navigation between user interfaces. In some embodiments, the touchpad, when touched by the user, navigates device 100 to a main, home, or root menu from any user interface that is displayed on device 100. In such embodiments, a “menu button” is implemented using a touchpad. In some other embodiments, the menu button is a physical push button or other physical input control device instead of a touchpad.

FIG. 1B is a block diagram illustrating exemplary components for event handling in accordance with some embodiments. In some embodiments, memory 102 (FIG. 1A) or 370 (FIG. 3) includes event sorter 170 (e.g., in operating system 126) and a respective application 136-1 (e.g., any of the aforementioned applications 137-151, 155, 380-390).

Event sorter 170 receives event information and determines the application 136-1 and application view 191 of application 136-1 to which to deliver the event information. Event sorter 170 includes event monitor 171 and event dispatcher module 174. In some embodiments, application 136-1 includes application internal state 192, which indicates the current application view(s) displayed on touch-sensitive display 112 when the application is active or executing. In some embodiments, device/global internal state 157 is used by event sorter 170 to determine which application(s) is (are) currently active, and application internal state 192 is used by event sorter 170 to determine application views 191 to which to deliver event information.

In some embodiments, application internal state 192 includes additional information, such as one or more of: resume information to be used when application 136-1 resumes execution, user interface state information that indicates information being displayed or that is ready for display by application 136-1, a state queue for enabling the user to go back to a prior state or view of application 136-1, and a redo/undo queue of previous actions taken by the user.

Event monitor 171 receives event information from peripherals interface 118. Event information includes information about a sub-event (e.g., a user touch on touch-sensitive display 112, as part of a multi-touch gesture). Peripherals interface 118 transmits information it receives from I/O subsystem 106 or a sensor, such as proximity sensor 166, accelerometer(s) 168, and/or microphone 113 (through audio circuitry 110). Information that peripherals interface 118 receives from I/O subsystem 106 includes information from touch-sensitive display 112 or a touch-sensitive surface.

In some embodiments, event monitor 171 sends requests to the peripherals interface 118 at predetermined intervals. In response, peripherals interface 118 transmits event information. In other embodiments, peripherals interface 118 transmits event information only when there is a significant event (e.g., receiving an input above a predetermined noise threshold and/or for more than a predetermined duration).

In some embodiments, event sorter 170 also includes a hit view determination module 172 and/or an active event recognizer determination module 173.

Hit view determination module 172 provides software procedures for determining where a sub-event has taken place within one or more views when touch-sensitive display 112 displays more than one view. Views are made up of controls and other elements that a user can see on the display.

Another aspect of the user interface associated with an application is a set of views, sometimes herein called application views or user interface windows, in which information is displayed and touch-based gestures occur. The application views (of a respective application) in which a touch is detected optionally correspond to programmatic levels within a programmatic or view hierarchy of the application. For example, the lowest level view in which a touch is detected is, optionally, called the hit view, and the set of events that are recognized as proper inputs are, optionally, determined based, at least in part, on the hit view of the initial touch that begins a touch-based gesture.

Hit view determination module 172 receives information related to sub-events of a touch-based gesture. When an application has multiple views organized in a hierarchy, hit view determination module 172 identifies a hit view as the lowest view in the hierarchy which should handle the sub-event. In most circumstances, the hit view is the lowest level view in which an initiating sub-event occurs (e.g., the first sub-event in the sequence of sub-events that form an event or potential event). Once the hit view is identified by the hit view determination module 172, the hit view typically receives all sub-events related to the same touch or input source for which it was identified as the hit view.

Active event recognizer determination module 173 determines which view or views within a view hierarchy should receive a particular sequence of sub-events. In some embodiments, active event recognizer determination module 173 determines that only the hit view should receive a particular sequence of sub-events. In other embodiments, active event recognizer determination module 173 determines that all views that include the physical location of a sub-event are actively involved views, and therefore determines that all actively involved views should receive a particular sequence of sub-events. In other embodiments, even if touch sub-events were entirely confined to the area associated with one particular view, views higher in the hierarchy would still remain as actively involved views.

Event dispatcher module 174 dispatches the event information to an event recognizer (e.g., event recognizer 180). In embodiments including active event recognizer determination module 173, event dispatcher module 174 delivers the event information to an event recognizer determined by active event recognizer determination module 173. In some embodiments, event dispatcher module 174 stores in an event queue the event information, which is retrieved by a respective event receiver 182.

In some embodiments, operating system 126 includes event sorter 170. Alternatively, application 136-1 includes event sorter 170. In yet other embodiments, event sorter 170 is a stand-alone module, or a part of another module stored in memory 102, such as contact/motion module 130.

In some embodiments, application 136-1 includes a plurality of event handlers 190 and one or more application views 191, each of which includes instructions for handling touch events that occur within a respective view of the application's user interface. Each application view 191 of the application 136-1 includes one or more event recognizers 180. Typically, a respective application view 191 includes a plurality of event recognizers 180. In other embodiments, one or more of event recognizers 180 are part of a separate module, such as a user interface kit or a higher level object from which application 136-1 inherits methods and other properties. In some embodiments, a respective event handler 190 includes one or more of: data updater 176, object updater 177, GUI updater 178, and/or event data 179 received from event sorter 170. Event handler 190 optionally utilizes or calls data updater 176, object updater 177, or GUI updater 178 to update the application internal state 192. Alternatively, one or more of the application views 191 include one or more respective event handlers 190. Also, in some embodiments, one or more of data updater 176, object updater 177, and GUI updater 178 are included in a respective application view 191.

A respective event recognizer 180 receives event information (e.g., event data 179) from event sorter 170 and identifies an event from the event information. Event recognizer 180 includes event receiver 182 and event comparator 184. In some embodiments, event recognizer 180 also includes at least a subset of: metadata 183, and event delivery instructions 188 (which optionally include sub-event delivery instructions).

Event receiver 182 receives event information from event sorter 170. The event information includes information about a sub-event, for example, a touch or a touch movement. Depending on the sub-event, the event information also includes additional information, such as location of the sub-event. When the sub-event concerns motion of a touch, the event information optionally also includes speed and direction of the sub-event. In some embodiments, events include rotation of the device from one orientation to another (e.g., from a portrait orientation to a landscape orientation, or vice versa), and the event information includes corresponding information about the current orientation (also called device attitude) of the device.

Event comparator 184 compares the event information to predefined event or sub-event definitions and, based on the comparison, determines an event or sub-event, or determines or updates the state of an event or sub-event. In some embodiments, event comparator 184 includes event definitions 186. Event definitions 186 contain definitions of events (e.g., predefined sequences of sub-events), for example, event 1 (187-1), event 2 (187-2), and others. In some embodiments, sub-events in an event (e.g., 187-1 and/or 187-2) include, for example, touch begin, touch end, touch movement, touch cancellation, and multiple touching. In one example, the definition for event 1 (187-1) is a double tap on a displayed object. The double tap, for example, comprises a first touch (touch begin) on the displayed object for a predetermined phase, a first liftoff (touch end) for a predetermined phase, a second touch (touch begin) on the displayed object for a predetermined phase, and a second liftoff (touch end) for a predetermined phase. In another example, the definition for event 2 (187-2) is a dragging on a displayed object. The dragging, for example, comprises a touch (or contact) on the displayed object for a predetermined phase, a movement of the touch across touch-sensitive display 112, and liftoff of the touch (touch end). In some embodiments, the event also includes information for one or more associated event handlers 190.

In some embodiments, event definitions 186 include a definition of an event for a respective user-interface object. In some embodiments, event comparator 184 performs a hit test to determine which user-interface object is associated with a sub-event. For example, in an application view in which three user-interface objects are displayed on touch-sensitive display 112, when a touch is detected on touch-sensitive display 112, event comparator 184 performs a hit test to determine which of the three user-interface objects is associated with the touch (sub-event). If each displayed object is associated with a respective event handler 190, the event comparator uses the result of the hit test to determine which event handler 190 should be activated. For example, event comparator 184 selects an event handler associated with the sub-event and the object triggering the hit test.

In some embodiments, the definition for a respective event (187) also includes delayed actions that delay delivery of the event information until after it has been determined whether the sequence of sub-events does or does not correspond to the event recognizer's event type.

When a respective event recognizer 180 determines that the series of sub-events do not match any of the events in event definitions 186, the respective event recognizer 180 enters an event impossible, event failed, or event ended state, after which it disregards subsequent sub-events of the touch-based gesture. In this situation, other event recognizers, if any, that remain active for the hit view continue to track and process sub-events of an ongoing touch-based gesture.

In some embodiments, a respective event recognizer 180 includes metadata 183 with configurable properties, flags, and/or lists that indicate how the event delivery system should perform sub-event delivery to actively involved event recognizers. In some embodiments, metadata 183 includes configurable properties, flags, and/or lists that indicate how event recognizers interact, or are enabled to interact, with one another. In some embodiments, metadata 183 includes configurable properties, flags, and/or lists that indicate whether sub-events are delivered to varying levels in the view or programmatic hierarchy.

In some embodiments, a respective event recognizer 180 activates event handler 190 associated with an event when one or more particular sub-events of an event are recognized. In some embodiments, a respective event recognizer 180 delivers event information associated with the event to event handler 190. Activating an event handler 190 is distinct from sending (and deferred sending) sub-events to a respective hit view. In some embodiments, event recognizer 180 throws a flag associated with the recognized event, and event handler 190 associated with the flag catches the flag and performs a predefined process.

In some embodiments, event delivery instructions 188 include sub-event delivery instructions that deliver event information about a sub-event without activating an event handler. Instead, the sub-event delivery instructions deliver event information to event handlers associated with the series of sub-events or to actively involved views. Event handlers associated with the series of sub-events or with actively involved views receive the event information and perform a predetermined process.

In some embodiments, data updater 176 creates and updates data used in application 136-1. For example, data updater 176 updates the telephone number used in contacts module 137, or stores a video file used in video player module. In some embodiments, object updater 177 creates and updates objects used in application 136-1. For example, object updater 177 creates a new user-interface object or updates the position of a user-interface object. GUI updater 178 updates the GUI. For example, GUI updater 178 prepares display information and sends it to graphics module 132 for display on a touch-sensitive display.

In some embodiments, event handler(s) 190 includes or has access to data updater 176, object updater 177, and GUI updater 178. In some embodiments, data updater 176, object updater 177, and GUI updater 178 are included in a single module of a respective application 136-1 or application view 191. In other embodiments, they are included in two or more software modules.

It shall be understood that the foregoing discussion regarding event handling of user touches on touch-sensitive displays also applies to other forms of user inputs to operate multifunction devices 100 with input devices, not all of which are initiated on touch screens. For example, mouse movement and mouse button presses, optionally coordinated with single or multiple keyboard presses or holds; contact movements such as taps, drags, scrolls, etc. on touchpads; pen stylus inputs; movement of the device; oral instructions; detected eye movements; biometric inputs; and/or any combination thereof are optionally utilized as inputs corresponding to sub-events which define an event to be recognized.

FIG. 2 illustrates a portable multifunction device 100 having a touch screen 112 in accordance with some embodiments. The touch screen optionally displays one or more graphics within user interface (UI) 200. In this embodiment, as well as others described below, a user is enabled to select one or more of the graphics by making a gesture on the graphics, for example, with one or more fingers 202 (not drawn to scale in the figure) or one or more styluses 203 (not drawn to scale in the figure). In some embodiments, selection of one or more graphics occurs when the user breaks contact with the one or more graphics. In some embodiments, the gesture optionally includes one or more taps, one or more swipes (from left to right, right to left, upward and/or downward), and/or a rolling of a finger (from right to left, left to right, upward and/or downward) that has made contact with device 100. In some implementations or circumstances, inadvertent contact with a graphic does not select the graphic. For example, a swipe gesture that sweeps over an application icon optionally does not select the corresponding application when the gesture corresponding to selection is a tap.

Device 100 optionally also include one or more physical buttons, such as “home” or menu button 204. As described previously, menu button 204 is, optionally, used to navigate to any application 136 in a set of applications that are, optionally, executed on device 100. Alternatively, in some embodiments, the menu button is implemented as a soft key in a GUI displayed on touch screen 112.

In some embodiments, device 100 includes touch screen 112, menu button 204, push button 206 for powering the device on/off and locking the device, volume adjustment button(s) 208, subscriber identity module (SIM) card slot 210, headset jack 212, and docking/charging external port 124. Push button 206 is, optionally, used to turn the power on/off on the device by depressing the button and holding the button in the depressed state for a predefined time interval; to lock the device by depressing the button and releasing the button before the predefined time interval has elapsed; and/or to unlock the device or initiate an unlock process. In an alternative embodiment, device 100 also accepts verbal input for activation or deactivation of some functions through microphone 113. Device 100 also, optionally, includes one or more contact intensity sensors 165 for detecting intensity of contacts on touch screen 112 and/or one or more tactile output generators 167 for generating tactile outputs for a user of device 100.

FIG. 3 is a block diagram of an exemplary multifunction device with a display and a touch-sensitive surface in accordance with some embodiments. Device 300 need not be portable. In some embodiments, device 300 is a laptop computer, a desktop computer, a tablet computer, a multimedia player device, a navigation device, an educational device (such as a child's learning toy), a gaming system, or a control device (e.g., a home or industrial controller). Device 300 typically includes one or more processing units (CPUs) 310, one or more network or other communications interfaces 360, memory 370, and one or more communication buses 320 for interconnecting these components. Communication buses 320 optionally include circuitry (sometimes called a chipset) that interconnects and controls communications between system components. Device 300 includes input/output (I/O) interface 330 comprising display 340, which is typically a touch screen display. I/O interface 330 also optionally includes a keyboard and/or mouse (or other pointing device) 350 and touchpad 355, tactile output generator 357 for generating tactile outputs on device 300 (e.g., similar to tactile output generator(s) 167 described above with reference to FIG. 1A), sensors 359 (e.g., optical, acceleration, proximity, touch-sensitive, and/or contact intensity sensors similar to contact intensity sensor(s) 165 described above with reference to FIG. 1A). Memory 370 includes high-speed random access memory, such as DRAM, SRAM, DDR RAM, or other random access solid state memory devices; and optionally includes non-volatile memory, such as one or more magnetic disk storage devices, optical disk storage devices, flash memory devices, or other non-volatile solid state storage devices. Memory 370 optionally includes one or more storage devices remotely located from CPU(s) 310. In some embodiments, memory 370 stores programs, modules, and data structures analogous to the programs, modules, and data structures stored in memory 102 of portable multifunction device 100 (FIG. 1A), or a subset thereof. Furthermore, memory 370 optionally stores additional programs, modules, and data structures not present in memory 102 of portable multifunction device 100. For example, memory 370 of device 300 optionally stores drawing module 380, presentation module 382, word processing module 384, website creation module 386, disk authoring module 388, and/or spreadsheet module 390, while memory 102 of portable multifunction device 100 (FIG. 1A) optionally does not store these modules.

Each of the above-identified elements in FIG. 3 is, optionally, stored in one or more of the previously mentioned memory devices. Each of the above-identified modules corresponds to a set of instructions for performing a function described above. The above-identified modules or computer programs (e.g., sets of instructions or including instructions) need not be implemented as separate software programs (such as computer programs (e.g., including instructions)), procedures, or modules, and thus various subsets of these modules are, optionally, combined or otherwise rearranged in various embodiments. In some embodiments, memory 370 optionally stores a subset of the modules and data structures identified above. Furthermore, memory 370 optionally stores additional modules and data structures not described above.

Attention is now directed towards embodiments of user interfaces that are, optionally, implemented on, for example, portable multifunction device 100.

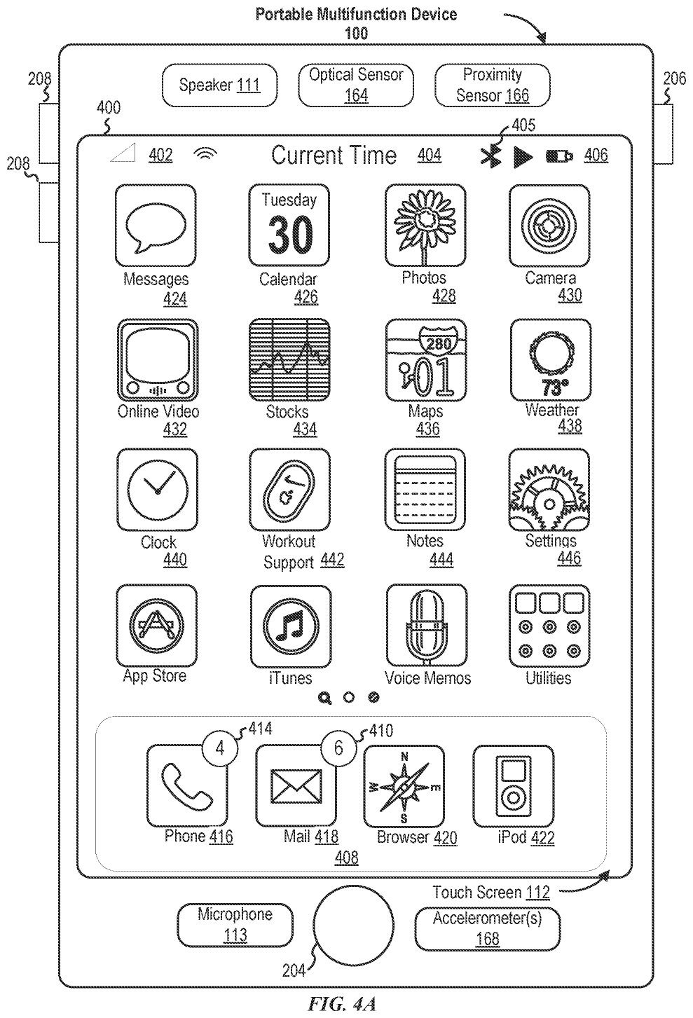

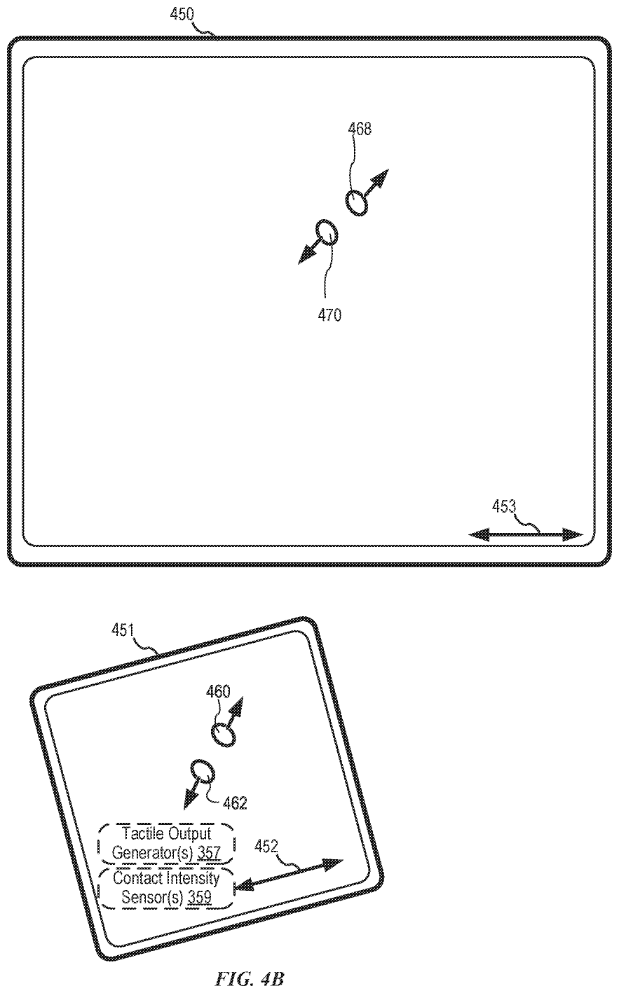

FIG. 4A illustrates an exemplary user interface for a menu of applications on portable multifunction device 100 in accordance with some embodiments. Similar user interfaces are, optionally, implemented on device 300. In some embodiments, user interface 400 includes the following elements, or a subset or superset thereof:
Signal strength indicator(s) 402 for wireless communication(s), such as cellular and Wi-Fi signals;
Time 404;
Bluetooth indicator 405;
Battery status indicator 406;
Tray 408 with icons for frequently used applications, such as:
   Icon 416 for telephone module 138, labeled “Phone,” which optionally includes an indicator 414 of the number of missed calls or voicemail messages;
   Icon 418 for e-mail client module 140, labeled “Mail,” which optionally includes an indicator 410 of the number of unread e-mails;
   Icon 420 for browser module 147, labeled “Browser;” and
   Icon 422 for video and music player module 152, also referred to as iPod (trademark of Apple Inc.) module 152, labeled “iPod;” and
Icons for other applications, such as:
   Icon 424 for IM module 141, labeled “Messages;”
   Icon 426 for calendar module 148, labeled “Calendar;”
   Icon 428 for image management module 144, labeled “Photos;”
   Icon 430 for camera module 143, labeled “Camera;”
   Icon 432 for online video module 155, labeled “Online Video;”
   Icon 434 for stocks widget 149-2, labeled “Stocks;”
   Icon 436 for map module 154, labeled “Maps;”
   Icon 438 for weather widget 149-1, labeled “Weather;”
   Icon 440 for alarm clock widget 149-4, labeled “Clock;”
   Icon 442 for workout support module 142, labeled “Workout Support;”
   Icon 444 for notes module 153, labeled “Notes;” and
   Icon 446 for a settings application or module, labeled “Settings,” which provides access to settings for device 100 and its various applications 136.

It should be noted that the icon labels illustrated in FIG. 4A are merely exemplary. For example, icon 422 for video and music player module 152 is labeled “Music” or “Music Player.” Other labels are, optionally, used for various application icons. In some embodiments, a label for a respective application icon includes a name of an application corresponding to the respective application icon. In some embodiments, a label for a particular application icon is distinct from a name of an application corresponding to the particular application icon.

FIG. 4B illustrates an exemplary user interface on a device (e.g., device 300, FIG. 3) with a touch-sensitive surface 451 (e.g., a tablet or touchpad 355, FIG. 3) that is separate from the display 450 (e.g., touch screen display 112). Device 300 also, optionally, includes one or more contact intensity sensors (e.g., one or more of sensors 359) for detecting intensity of contacts on touch-sensitive surface 451 and/or one or more tactile output generators 357 for generating tactile outputs for a user of device 300.

Although some of the examples that follow will be given with reference to inputs on touch screen display 112 (where the touch-sensitive surface and the display are combined), in some embodiments, the device detects inputs on a touch-sensitive surface that is separate from the display, as shown in FIG. 4B. In some embodiments, the touch-sensitive surface (e.g., 451 in FIG. 4B) has a primary axis (e.g., 452 in FIG. 4B) that corresponds to a primary axis (e.g., 453 in FIG. 4B) on the display (e.g., 450). In accordance with these embodiments, the device detects contacts (e.g., 460 and 462 in FIG. 4B) with the touch-sensitive surface 451 at locations that correspond to respective locations on the display (e.g., in FIG. 4B, 460 corresponds to 468 and 462 corresponds to 470). In this way, user inputs (e.g., contacts 460 and 462, and movements thereof) detected by the device on the touch-sensitive surface (e.g., 451 in FIG. 4B) are used by the device to manipulate the user interface on the display (e.g., 450 in FIG. 4B) of the multifunction device when the touch-sensitive surface is separate from the display. It should be understood that similar methods are, optionally, used for other user interfaces described herein.

Additionally, while the following examples are given primarily with reference to finger inputs (e.g., finger contacts, finger tap gestures, finger swipe gestures), it should be understood that, in some embodiments, one or more of the finger inputs are replaced with input from another input device (e.g., a mouse-based input or stylus input). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

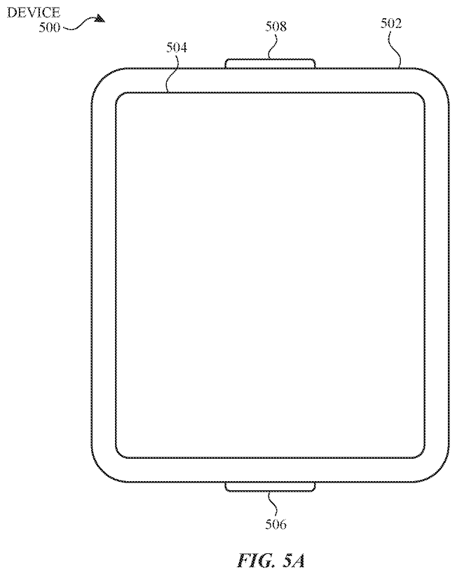

FIG. 5A illustrates exemplary personal electronic device 500. Device 500 includes body 502. In some embodiments, device 500 can include some or all of the features described with respect to devices 100 and 300 (e.g., FIGS. 1A-4B). In some embodiments, device 500 has touch-sensitive display screen 504, hereafter touch screen 504. Alternatively, or in addition to touch screen 504, device 500 has a display and a touch-sensitive surface. As with devices 100 and 300, in some embodiments, touch screen 504 (or the touch-sensitive surface) optionally includes one or more intensity sensors for detecting intensity of contacts (e.g., touches) being applied. The one or more intensity sensors of touch screen 504 (or the touch-sensitive surface) can provide output data that represents the intensity of touches. The user interface of device 500 can respond to touches based on their intensity, meaning that touches of different intensities can invoke different user interface operations on device 500.

Exemplary techniques for detecting and processing touch intensity are found, for example, in related applications: International Patent Application Serial No. PCT/US2013/040061, titled “Device, Method, and Graphical User Interface for Displaying User Interface Objects Corresponding to an Application,” filed May 8, 2013, published as WIPO Publication No. WO/2013/169849, and International Patent Application Serial No. PCT/US2013/069483, titled “Device, Method, and Graphical User Interface for Transitioning Between Touch Input to Display Output Relationships,” filed Nov. 11, 2013, published as WIPO Publication No. WO/2014/105276, each of which is hereby incorporated by reference in their entirety.

In some embodiments, device 500 has one or more input mechanisms 506 and 508. Input mechanisms 506 and 508, if included, can be physical. Examples of physical input mechanisms include push buttons and rotatable mechanisms. In some embodiments, device 500 has one or more attachment mechanisms. Such attachment mechanisms, if included, can permit attachment of device 500 with, for example, hats, eyewear, earrings, necklaces, shirts, jackets, bracelets, watch straps, chains, trousers, belts, shoes, purses, backpacks, and so forth. These attachment mechanisms permit device 500 to be worn by a user.

FIG. 5B depicts exemplary personal electronic device 500. In some embodiments, device 500 can include some or all of the components described with respect to FIGS. 1A, 1, and 3. Device 500 has bus 512 that operatively couples I/O section 514 with one or more computer processors 516 and memory 518. I/O section 514 can be connected to display 504, which can have touch-sensitive component 522 and, optionally, intensity sensor 524 (e.g., contact intensity sensor). In addition, I/O section 514 can be connected with communication unit 530 for receiving application and operating system data, using Wi-Fi, Bluetooth, near field communication (NFC), cellular, and/or other wireless communication techniques. Device 500 can include input mechanisms 506 and/or 508. Input mechanism 506 is, optionally, a rotatable input device or a depressible and rotatable input device, for example. Input mechanism 508 is, optionally, a button, in some examples.

Input mechanism 508 is, optionally, a microphone, in some examples. Personal electronic device 500 optionally includes various sensors, such as GPS sensor 532, accelerometer 534, directional sensor 540 (e.g., compass), gyroscope 536, motion sensor 538, and/or a combination thereof, all of which can be operatively connected to I/O section 514.

Memory 518 of personal electronic device 500 can include one or more non-transitory computer-readable storage mediums, for storing computer-executable instructions, which, when executed by one or more computer processors 516, for example, can cause the computer processors to perform the techniques described below, including process 700 (FIG. 7). A computer-readable storage medium can be any medium that can tangibly contain or store computer-executable instructions for use by or in connection with the instruction execution system, apparatus, or device. In some examples, the storage medium is a transitory computer-readable storage medium. In some examples, the storage medium is a non-transitory computer-readable storage medium. The non-transitory computer-readable storage medium can include, but is not limited to, magnetic, optical, and/or semiconductor storages. Examples of such storage include magnetic disks, optical discs based on CD, DVD, or Blu-ray technologies, as well as persistent solid-state memory such as flash, solid-state drives, and the like. Personal electronic device 500 is not limited to the components and configuration of FIG. 5B, but can include other or additional components in multiple configurations.

As used here, the term “affordance” refers to a user-interactive graphical user interface object that is, optionally, displayed on the display screen of devices 100, 300, and/or 500 (FIGS. 1A, 3, and 5A-5B). For example, an image (e.g., icon), a button, and text (e.g., hyperlink) each optionally constitute an affordance.

As used herein, the term “focus selector” refers to an input element that indicates a current part of a user interface with which a user is interacting. In some implementations that include a cursor or other location marker, the cursor acts as a “focus selector” so that when an input (e.g., a press input) is detected on a touch-sensitive surface (e.g., touchpad 355 in FIG. 3 or touch-sensitive surface 451 in FIG. 4B) while the cursor is over a particular user interface element (e.g., a button, window, slider, or other user interface element), the particular user interface element is adjusted in accordance with the detected input. In some implementations that include a touch screen display (e.g., touch-sensitive display system 112 in FIG. 1A or touch screen 112 in FIG. 4A) that enables direct interaction with user interface elements on the touch screen display, a detected contact on the touch screen acts as a “focus selector” so that when an input (e.g., a press input by the contact) is detected on the touch screen display at a location of a particular user interface element (e.g., a button, window, slider, or other user interface element), the particular user interface element is adjusted in accordance with the detected input. In some implementations, focus is moved from one region of a user interface to another region of the user interface without corresponding movement of a cursor or movement of a contact on a touch screen display (e.g., by using a tab key or arrow keys to move focus from one button to another button); in these implementations, the focus selector moves in accordance with movement of focus between different regions of the user interface. Without regard to the specific form taken by the focus selector, the focus selector is generally the user interface element (or contact on a touch screen display) that is controlled by the user so as to communicate the user's intended interaction with the user interface (e.g., by indicating, to the device, the element of the user interface with which the user is intending to interact). For example, the location of a focus selector (e.g., a cursor, a contact, or a selection box) over a respective button while a press input is detected on the touch-sensitive surface (e.g., a touchpad or touch screen) will indicate that the user is intending to activate the respective button (as opposed to other user interface elements shown on a display of the device).

As used in the specification and claims, the term “characteristic intensity” of a contact refers to a characteristic of the contact based on one or more intensities of the contact. In some embodiments, the characteristic intensity is based on multiple intensity samples. The characteristic intensity is, optionally, based on a predefined number of intensity samples, or a set of intensity samples collected during a predetermined time period (e.g., 0.05, 0.1, 0.2, 0.5, 1, 2, 5, 10 seconds) relative to a predefined event (e.g., after detecting the contact, prior to detecting liftoff of the contact, before or after detecting a start of movement of the contact, prior to detecting an end of the contact, before or after detecting an increase in intensity of the contact, and/or before or after detecting a decrease in intensity of the contact). A characteristic intensity of a contact is, optionally, based on one or more of: a maximum value of the intensities of the contact, a mean value of the intensities of the contact, an average value of the intensities of the contact, a top 10 percentile value of the intensities of the contact, a value at the half maximum of the intensities of the contact, a value at the 90 percent maximum of the intensities of the contact, or the like. In some embodiments, the duration of the contact is used in determining the characteristic intensity (e.g., when the characteristic intensity is an average of the intensity of the contact over time). In some embodiments, the characteristic intensity is compared to a set of one or more intensity thresholds to determine whether an operation has been performed by a user. For example, the set of one or more intensity thresholds optionally includes a first intensity threshold and a second intensity threshold. In this example, a contact with a characteristic intensity that does not exceed the first threshold results in a first operation, a contact with a characteristic intensity that exceeds the first intensity threshold and does not exceed the second intensity threshold results in a second operation, and a contact with a characteristic intensity that exceeds the second threshold results in a third operation. In some embodiments, a comparison between the characteristic intensity and one or more thresholds is used to determine whether or not to perform one or more operations (e.g., whether to perform a respective operation or forgo performing the respective operation), rather than being used to determine whether to perform a first operation or a second operation.

Attention is now directed towards embodiments of user interfaces (“UI”) and associated processes that are implemented on an electronic device, such as portable multifunction device 100, device 300, or device 500.

FIGS. 6A-6M illustrate exemplary user interfaces including information related to physiological measurements, in accordance with some embodiments. The user interfaces in these figures are used to illustrate the processes described below, including the processes in FIG. 7.

FIG. 6A illustrates device 600, which includes display 602 (e.g., a touchscreen display or a non-touchscreen display) in a low-power state (e.g., a display off state and/or a state that uses reduced power as compared to a non-low-power state). Device 600 is a smart phone having one or more features of devices 100, 300, and/or 500 and can be used for tracking health data of a user. In some embodiments, device 600 includes sensors (e.g., accelerometer, gyroscope, GPS sensor, heart rate sensor, and/or blood oxygen sensor) for tracking health-related user data. In some embodiments, device 600 is in communication with one or more smart devices (e.g., a smart watch and/or a heart rate monitor) via one or more wireless communication protocols (e.g., Bluetooth, WiFi, and/or an ultra-wideband connection), and the one or more smart devices are associated with the user of device 600. In some embodiments, device 600 and the one or more smart devices are all associated with (e.g., logged into and/or using) the same user account of the same service. In the present example, device 600 is in communication with external device 604, which includes display 606 (e.g., a touchscreen display or non-touchscreen display). External device 604 is a smart watch having one or more features of device 600 and can be used for collecting health data of the user. In some embodiments, external device 604 includes sensors (e.g., accelerometer, gyroscope, GPS, optical heart sensor, electrical heart sensor, and/or blood oxygen sensor) for measuring and tracking physiological user data.

As shown in FIG. 6A, external device 604 is currently operating a workout application (e.g., Workout) (e.g., the workout application is an active application on external device 604). External device 604 includes display 606 displaying run workout user interface 608 for the active application. External device 604 is starting to record various metrics (e.g., distance and/or time) and health data (e.g., heart rate and/or non-heart rate data) of the user at the beginning of a workout, e.g., an outdoor run workout. In some embodiments, external device 604 also records (e.g., automatically and/or based on user requests) health data of the user during other types of activities (e.g., hiking, walking, biking, swimming, playing sports, dancing, and/or practicing yoga). In some embodiments, external device 604 records health data of the user without the workout application being active (e.g., the workout application is a closed application on external device 604) and/or without displaying a user interface of the workout application.

In FIG. 6A, run workout user interface 608 includes elapsed time 608a at “00:00.01,” as well as heart rate 610, pace 608b, and distance 608c at “0 FT.” External device 604 displays heart rate 610 and pace 608b in pending states (e.g., “- - BPM”) until enough user data is collected, via one or more sensors, to present a value for heart rate 610 and pace 608b, as shown in FIG. 6B.

In FIG. 6B, external device 604 continues recording the outdoor run workout in the workout application, as shown by run workout user interface 608 on display 606. Elapsed time 608a now reads “30:00.00,” indicating that 30 minutes has passed since the start of recording of the outdoor run workout. External device 604 has collected enough user data via the one or more sensors, so pace 608b now reads “8′34″ AVG MILE” to indicate that external device 604 has recorded the user running at an average pace of 8 minutes and 34 seconds per mile. Distance 608c now reads “3.50 MI” to indicate that external device 604 has recorded, via the one or more sensors, the user running for 3.5 miles. In some embodiments, distance is recorded via one or more sensors of device 600 and transmitted via one or more wireless communication protocols for display on display 606 of external device 604.

In FIG. 6B, run workout user interface 608 additionally includes run heart rate 610a, updated from heart rate 610 in FIG. 6A. External device 604 has collected enough user data via the one or more sensor of external device 604, so run heart rate 610a now reads “150 BPM.” In some embodiments, run heart rate 610a is the current heart rate of the user detected by external device 604. In some embodiments, run heart rate 610a is the average heart rate of the user during the outdoor run as detected and calculated by external device 604 over the 30 minute period. In some embodiments, run heart rate 610a is collected by an external sensor and transmitted via one or more wireless communication protocols to external device 604 for display on display 606. At FIG. 6B, external device 604 detects input 612 (e.g., a swipe input) on display 606, and in response to detecting input 612, external device 604 displays workout options user interface 614 on display 606, as shown in FIG. 6C.

In FIG. 6C, while external device 604 displays, on display 606, workout options user interface 614. Workout options user interface 614 includes the following: new workout affordance 614a to start recording of a new workout; pause affordance 614b to pause recording of the current outdoor run workout; end affordance 614c to end recording of the current outdoor run workout; and water lock affordance 614d to disable detection of inputs (e.g., touch inputs and/or water droplets that could be misinterpreted as an deliberate user input) on touchscreen display 606 of external device 604.

As shown in FIG. 6C, external device 604 detects input 616 (e.g., a tap input or press-and-hold input) at new workout affordance 614a. In some embodiments, in response to detecting input 616, external device 604 displays an additional user interface with various affordances (e.g., an outdoor walk affordance and an indoor walk affordance) corresponding to various workout types that can be selected (e.g., via touch input) to initiate recording of a new workout. In some embodiments, external device 604 detects an input selecting an outdoor walk affordance and, in response to detecting the selection of the outdoor walk affordance, begins recording various metrics (e.g., distance; time) and health data (e.g., heart rate) of the user at the beginning of an outdoor walk workout, as shown in FIG. 6D.

In FIG. 6D, while external device 604 is recording an outdoor walk workout in the workout application, external device 604 displays, on display 606, walk workout user interface 618. Walk workout user interface 618 and the various metrics contained within (e.g., elapsed time 618a, pace 618b, distance 618c, and walk heart rate 610b) are analogous in behavior to outdoor run workout user interface 608 and the various metrics contained within (e.g., elapsed time 608a, pace 608b, distance 608c, and run heart rate 610a).

In FIG. 6D, walk workout user interface 618 includes elapsed time 618a showing “01:00.00” to indicate that external device 604 has been recording the outdoor walk workout for 1 minute. Walk workout user interface 618 additionally includes walk heart rate 610b that reads “135 BPM.” In some embodiments, walk heart rate 610b is the current heart rate of the user detected by external device 604. In some embodiments, walk heart rate 610b is the average heart rate of the user during the outdoor walk as detected and calculated by external device 604 over the 1 minute period. In some embodiments, walk heart rate 610b is collected by an external sensor and transmitted via one or more wireless communication protocols to external device 604 for display on display 606.

From FIG. 6D to FIG. 6E, 30 seconds have passed, as indicated by elapsed time 618a showing “01:30.00” on walk workout user interface 618 to indicate that external device 604 has been recording the outdoor walk workout for 1 minute and 30 seconds. Pace 618b has remained constant between FIG. 6D and FIG. 6E. Distance 618c now reads “370 FT” to indicate that external device 604 has recorded, via the one or more sensors, the user walking for 370 feet. From FIG. 6D to FIG. 6E, walk heart rate 610b of “135 BPM” has decreased to walk heart rate 610c of “130 BPM.” In some embodiments, walk heart rate 610c is the current heart rate of the user detected by external device 604. In some embodiments, walk heart rate 610c is the average heart rate of the user during the outdoor walk as detected and calculated by external device 604 over the 1 minute and 30 second period. In some embodiments, walk heart rate 610c is collected by an external sensor and transmitted via one or more wireless communication protocols to external device 604 for display on display 606.

In some embodiments, external device 604 detects an input (e.g., a swipe input and/or an input analogous to input 612 of FIG. 6B) to navigate to a workout options user interface analogous to 614 of FIG. 6C. In some embodiments, external device 604 detects an input (e.g., a tap input and/or a press-and-hold input) on an end workout affordance analogous to end affordance 614c to end recording of the current outdoor walk workout. After ending recording of the current workout, external device 604 displays, on display 606, workout summary user interface 620, as shown in FIG. 6F. In some embodiments, device 600 and/or external device 604 automatically detect the beginning and/or ending of a workout based on movement of the respective devices and without requiring a user to specify to start and/or end the workout. In some embodiments, device 600 and/or external device 604 automatically determine the type of workout (e.g., outdoor run, indoor run, hiking, outdoor walk, indoor walk, and/or yoga) without requiring a user to input the type of workout. In some embodiments, device 600 and/or external device 604 automatically record heart rate and/or physical exertion information during automatically detected workouts.

In FIG. 6F, external device 604 displays, on display 606, workout summary user interface 620. Workout summary user interface 620 includes selectable outdoor run workout affordance 620a, selectable outdoor walk workout affordance 620b, total time 620c (e.g., “35:00.00;” the sum of 624a and 626a as shown on display 602 of device 600), active calories 620d (e.g., “400 CAL;” the sum of 624c and 626c as shown on display 602 of device 600), total calories 620e (e.g., “480 CAL;” the sum of 624d and 626d as shown on display 602 of device 600), and selectable done affordance 620f. In some embodiments, external device 604 detects an input (e.g., a tap input or a tap-and-hold input) at selectable outdoor run workout affordance 620a and in response, displays an outdoor run summary user interface including metrics (e.g., 608a-608c and 610a of FIG. 6B) recorded during the outdoor run portion of the user's workout. In some embodiments, external device 604 detects an input (e.g., a tap input or a tap-and-hold input) at selectable outdoor walk workout affordance 620b and in response, displays an outdoor walk summary user interface including metrics (e.g., 618a-618c and 610b of FIG. 6D; 618a-618c and 610c of FIG. 6E) recorded during the outdoor walk portion of the user's workout.

In some embodiments, external device 604 detects an input (e.g., a tap input or a tap-and-hold input) at selectable done affordance 620f and in response, ceases display of workout summary user interface 620. In some embodiments, external device 604 detects a set of one or more inputs (e.g., one or more tap inputs; one or more swipe inputs; a combination of tap inputs and swipe inputs) and in response to detecting the set of one or more inputs, ceases display of workout summary user interface 620 on display 606 and displays heart rate summary user interface 628, as shown in FIG. 6G. In some embodiments, external device 604 transmits the recorded workout data to device 600 for display on display 602.

Turning now to device 600 in FIG. 6F, display 602 of device 600 is no longer in the low-power mode (e.g., is instead in a high-power mode or a standard power mode). Device 600 displays, on display 602, workout summary user interface 622. Workout summary user interface 622 includes outdoor run section 624, corresponding to the outdoor run portion of the user's workout, and outdoor walk section 626, corresponding to the outdoor walk portion of the user's workout. Outdoor run section 624 includes total time 624a (e.g., “30:00.00;” 608a of FIG. 6B), distance 624b (e.g., “3.50 MI;” 608c of FIG. 6B), active calories 624c (e.g., “380 CAL”), total calories 624d (e.g., “445 CAL”), average heart rate 624e (e.g., “150 BPM”), average pace 624f (e.g., “8′34″/MI;” 608b of FIG. 6B), and heart rate graph 624g. Heart rate graph 624g shows the heart rate values recorded by one or more sensors on external device 604 during the outdoor run workout. In some embodiments, average heart rate 624e is the same heart rate measurement as heart rate 610a of FIG. 6B. In some embodiments, heart rate 610a of FIG. 6B is the current heart rate at the time of detection of heart rate by one or more sensors on external device 604, and therefore, average heart rate 624e as shown in FIG. 6F is not the same as heart rate 610a of FIG. 6B.

Additionally in FIG. 6F, outdoor walk section 626 includes total time 626a (e.g., “05:00.00”), distance 626b (e.g., “0.25 MI”), active calories 626c (e.g., “20 CAL”), total calories 626d (e.g., “35 CAL”), average heart rate 626e (e.g., “130 BPM”), average pace 626f (e.g., “20′05″/MI;” 618b of FIG. 6B). In some embodiments, outdoor walk section 626 includes a heart rate graph corresponding to the outdoor walk portion of the user's workout, analogous to heart rate graph 624g of outdoor run section 624. In some embodiments, device 600 detects an input (e.g., a swipe up input or a tap input) and in response, scrolls workout summary user interface 622 up to display the heart rate graph corresponding to the outdoor walk portion of the workout.

In some embodiments, device 600 detects a set of one or more inputs (e.g., one or more tap inputs; one or more swipe inputs; a combination of tap inputs and swipe inputs) and in response to detecting the set of one or more inputs, ceases display of workout summary user interface 622 on display 602 and displays health summary user interface 630, as shown in FIG. 6G.

In FIG. 6G, external device 604 displays, on display 606, heart rate summary user interface 628. Heart rate summary user interface 628 includes various selectable user interface objects for heart rate values (e.g., 628a-628g). In some embodiments, external device 604 detects an input (e.g., a tap input) corresponding to selection of one of the selectable user interface objects (e.g., 628a-628g) and in response, displays, on display 606, a detailed heart rate user interface with a graph similar to heart rate graph 624g of FIG. 6F.

In particular, in FIG. 6G, heart rate summary user interface 628 includes outdoor run heart rate affordance 628e (e.g., “150 BPM;” 624e of FIG. 6F), corresponding to the average heart rate recorded by the one or more sensors of external device 604 during the outdoor run portion of the workout as discussed with respect to FIG. 6A and FIG. 6B. Heart rate summary user interface 628 also includes outdoor walk heart rate affordance 628f (e.g., “130 BPM;” 626e of FIG. 6F), corresponding to the average heart rate recorded by the one or more sensors of external device 604 during the outdoor walk portion of the workout as discussed with respect to FIG. 6D and FIG. 6E. Heart rate summary user interface 628 also includes post workout heart rate affordance 628g (e.g., “120-80 BPM”), which is based on the decrease in heart rate in the three minutes following completion of the workouts (e.g., following the outdoor walk). Heart rate summary user interface 628 further includes recovery heart rate affordance 628c (e.g., “25 BPM”), which will be discussed in greater detail with respect to FIG. 6H.

Turning now to device 600 in FIG. 6G, device 600 displays, on display 602, health summary user interface 630, which is part of a health application (e.g., Health App) used for storing and reviewing health-related user data. Health summary user interface 630 includes various selectable user interface objects (e.g., 630a-630f). Activity summary affordance 630a includes metrics for calories, exercise minutes, and number of stand hours. In some embodiments, the calories and minutes of exercise include the calories burned and time recorded by external device 604 during the workout discussed with respect to FIGS. 6A-6F. Heart rate affordance 630b is analogous to heart rate affordance 628a shown on external device 604. Resting heart rate affordance 630c is analogous to resting heart rate affordance 628b shown on external device 604. Walking heart rate average affordance 630d is analogous to walking heart rate average affordance 628c shown on external device 604. Heart rate recovery affordance 630e is analogous to recovery heart rate affordance 628c shown on external device 604. Health summary user interface 630 also includes show all affordance 630f. In some embodiments, device 600 detects an input (e.g., a tap input or a tap-and-hold input) corresponding to selection of show all affordance 630f and in response, displays, on display 602, a list user interface with various selectable affordances corresponding to various health data types.

Health summary user interface 630 in FIG. 6G further includes selectable workout heart rate highlight 632 and selectable post workout heart rate highlight 634. Workout heart rate highlight 632 provides information corresponding to heart rate range as recording during the outdoor run portion of the workout, as discussed earlier with respect to FIGS. 6A-6B. In some embodiments, device 600 receives an input (e.g., a tap input or a tap-and-hold input) corresponding to selection of workout heart rate highlight 632, and in response, displays a heart rate user interface with additional details relating to heart rate measurements. Post workout heart rate highlight 634 provides information corresponding to heart rate measurements collected in the three minutes after completion of the recently recorded workout (e.g., the workouts discussed with respect to FIGS. 6A-6F). In some embodiments, device 600 receives an input (e.g., a tap input) corresponding to selection of post workout heart rate highlight 634, and in response, displays a heart rate user interface with additional details relating to heart rate measurements.

In FIG. 6G, device 600 detects input 636 (e.g., a tap input or a tap-and-hold input) corresponding to selection of heart rate recovery affordance 630e and, in response, displays heart rate recovery user interface 638 on display 602, as shown in FIG. 6H. Heart rate recovery user interface 638 includes graph region 638a. Graph region 638a includes heart rate recovery graph 640, heart rate recovery data points 640a-640g, selectable time period affordance 640h set to a week-long view, as indicated by the rectangle around “W,” and selectable trend affordance 640i. Heart rate recovery graph 640 shows seven heart rate recovery data points 640a-640g for the week of May 2-8, corresponding to the current day (e.g., Sunday, May 8) and previous six days respectively. Heart rate recovery data point 640a, for the current day (e.g., Sunday) reads 25, corresponding to “25 BPM” shown on heart rate recovery affordance 630e in FIG. 6G. In some embodiments, a heart rate recovery value (e.g., “25 BPM;” the values associated with heart rate recovery data points 640a-640g) is a prediction of a decrease in heart rate at one minute after ceasing physical activity after maximum physical exertion. In some embodiments, the values associated with heart rate recovery data points 640a-640g are not measured heart rate recovery values (e.g., they are not the difference between heart rate during physical activity (e.g., at maximum physical exertion) and at one minute after ceasing physical activity). As shown by heart rate recovery data points 640a-640g in heart rate recovery graph 640, the heart rate recovery value can vary from day to day (e.g., heart rate recovery data points 640a, 640c, 640d, and 640g are 25 BPM, while heart rate recovery data point 640b is 28 BPM and heart rate recover data point 640f is 24 BPM). In some embodiments, heart rate recovery values are predicted based on a set of one or more recorded heart rate values recorded during one or more types of physical activity (e.g., running, walking, hiking, swimming, stretching, resting). In some embodiments, heart rate recovery values are predicted (and/or displayed) by device 600. In some embodiments, heart rate recovery values are predicted (and/or displayed) by external device 604. In some embodiments, heart rate recovery values are higher than an actual change in heart rate between a first exercise (e.g., running or a first physical activity) and a second exercise (e.g., walking or a second physical activity). In some embodiments, the heart rate recovery values are not based on a heart rate detected during maximum physical exertion (e.g., the heart rate recovery uses a heart rate detected during non-maximum physical exertion to predict the heart rate recovery). In some embodiments, heart rate recovery values are lower than the actual change in heart rate between a first exercise (e.g., running) and a second exercise (e.g., walking). Thus, the displayed heart rate recovery values are predicted values based on a first exercise (e.g., running) and a second exercise (e.g., walking, and/or an exercise that is not resting), rather than an actual measure of heart rate recovery one minute after ending physical activity (e.g., ending a maximum physical exertion or ending a non-maximum physical exertion). In some embodiments, the heart rate recover value is different from a difference between a heart rate measured at the end of the first exercise and a heart rate measured at one minute after the end of the first exercise (e.g., one minute into the second exercise). In some embodiments, the heart rate recovery value is not based on a heart rate measured while the user is not exercising. In some embodiments, the user is not required to cease exercising or cease physical activity in order for a heart rate recovery value (which predicts what the user's actual heart rate recovery would be had the user ceased exercising and/or ceased physical activity) to be determined and subsequently displayed. Thus, the user can proceed to exercise as they prefer and be able to review their heart rate recovery values without needing to perform a first exercise (e.g., at maximum or non-maximum physical exertion) following by ceasing exercising (e.g., for one minute or for three minutes).

In some embodiments, in response to selection of a different time period within time period affordance 640h (e.g., “D;” “M”), device 600 updates heart rate recovery graph 640 from a week-long view of heart rate recovery data points, as shown in FIG. 6H, to a different view of heart rate recovery data points (e.g., a day of data points if “D” is selected; a month of data points if “M” is selected; six months of data points if “6 M” is selected; a year of data points if “Y” is selected). In some embodiments, selection of a longer time period in time period affordance 640h (e.g., “M;” “6 M;” “Y”) causes display of heart rate recovery value averages within heart rate recovery graph 640. In FIG. 6H, selectable trend affordance 640i reads, “UNAVAILABLE”. In some embodiments, more than a minimum number of data points are required for device 600 to calculate a trend in data (e.g., more than one week). In some embodiments, selection of a longer time period in time period affordance 640h (e.g., “M;” “6 M;” “Y”) causes selectable trend affordance 640i to update from “UNAVAILABLE” in FIG. 6H to a trend value (e.g., a consistent average heart rate recovery value, an increase or decrease in heart rate recovery value) that can be selected to cause display of a trend line on heart rate recovery graph 640.

In FIG. 6H, heart rate recovery user interface 638 further includes selectable about affordance 638b, selectable favorites affordance 638c, selectable show all data affordance 638d, selectable data sources affordance 638e, and unit affordance 638f to indicate the unit of measurement for heart rate (e.g., “BPM”). In some embodiments, device 600 detects an input (e.g., a tap input or a tap-and-hold input) corresponding to selection of about affordance 638b and in response, displays a user interface with explanatory details relating to heart rate recovery on display 602. In some embodiments, device 600 detects an input (e.g., a tap input or a tap-and-hold input) corresponding to selection of favorites affordance 638c and in response, provides a visual indication of receiving the selection (e.g., by updating the star from filled to unfilled) and removes heart rate recovery affordance 630e from being displayed within health summary user interface 630 of FIG. 6G. In some embodiments, device 600 detects an input (e.g., a tap input or a tap-and-hold input) corresponding to selection of data sources affordance 638e and in response, displays a user interface detailing one or more devices (e.g., device 600; external device 604) used for recording heart rate data and permission settings for other applications accessing recorded heart rate data. In FIG. 6H, device 600 detects input 642 (e.g., a tap input or a tap-and-hold input) corresponding to selection of show all data affordance 638d and in response, displays all data user interface 644, as shown in FIG. 6I.

In FIG. 6I, device 600 displays recorded data user interface 644 on display 602. Recorded data user interface 644 includes selectable heart rate recovery entries 644a-644j. In some embodiments, device 600 detects a swipe input on display 602 and in response, scrolls recorded data user interface 644 to show additional selectable heart rate recovery entries. Device 600 detects first input 646a corresponding to selection of heart rate recovery entry 644a and in response, displays details user interface 648a on display 602, as shown in FIG. 6J. Device 600 detects second input 646b corresponding to selection of heart rate recovery entry 644c and in response, displays details user interface 648b on display 602, as shown in FIG. 6K. Device 600 detects third input 646c corresponding to selection of heart rate recovery entry 644d and in response, displays details user interface 648c on display 602, as shown in FIG. 6L. Device 600 detects fourth input 646d corresponding to selection of heart rate recovery entry 644f and in response, displays details user interface 648d on display 602, as shown in FIG. 6M.

In FIG. 6J, device 600 displays details user interface 648a on display 602 in response to first input 646a in FIG. 6I. Details user interface 648a includes heart rate recovery value 650a, date 652a, source 654a, date added to health 656a, activity type 658a, recovery behavior 660a, medication factor 662a, and device details section 664a. In FIG. 6J, heart rate recovery value 650a is “25 BPM.” Heart rate recovery value 650a was generated on “MAY 8, 2022 AT 10:45 AM,” as indicated within date 652a. Heart rate data used for determining heart recovery value 650a was collected using “JANE APPLESEED'S WATCH,” (e.g., external device 604) as indicated by source 654a. More details about source 654a are included in device details section 664a. Heart rate recovery value 650a was added to the health application (e.g., Health App) on “MAY 8, 2022 AT 10:45 AM,” as indicated within date added to health 656a. In this example, date added to health 656a is the same as date 652a. In some embodiments, date added to health 656a is the different from date 652a, such as when the source device (e.g., external device 604) is not in communication with device 600 and later regains communication. The physical activities in which heart rate data was collected for use in determining heart recovery value 650a are indicated by activity type 658a (e.g., “OUTDOOR RUN”) and recovery behavior 660a (e.g., “OUTDOOR WALK”). Medication factor 662a indicates that a medication affecting heart rate was not used during heart rate data collection for determining heart rate recovery value 650a. In some embodiments, additional factors that affect heart rate (e.g., caffeine intake) are included in details user interface 648a.

In FIG. 6K, device 600 displays details user interface 648b on display 602 in response to second input 646b in FIG. 6I. Details user interface 648b includes heart rate recovery value 650b, date 652b, source 654b, date added to health 656b, activity type 658b, recovery behavior 660b, medication factor 662b, and device details 664b, which are analogous to 650a-664a of details user interface 648a as described in detail with respect to FIG. 6J. In particular, heart rate recovery value 650b is “25 BPM,” the same as heart rate recovery value 650a of FIG. 6J. The physical activities in which heart rate data was collected for use in determining heart recovery value 650b are indicated by activity type 658b (e.g., “OUTDOOR RUN”) and recovery behavior 660b (e.g., “OUTDOOR WALK”). Activity type 658b and recovery behavior 660b are the same as activity type 658a and recovery behavior 660a of FIG. 6J, “OUTDOOR RUN” and “OUTDOOR WALK,” respectively. Heart rate recovery value 650b was generated on “MAY 6, 2022 AT 3:21 PM,” as indicated within date 652b, a different day from date 652a of FIG. 6J. Despite being generated on different days, heart rate recovery value 650b of FIG. 6K and heart rate recovery value 650a of FIG. 6J are the same, reading “25 BPM.” In some embodiments, the same activity type (e.g., 658a; 658b) and same recovery type (e.g., 660a; 660b) recorded on different days (e.g., 652a; 652b) result in different heart rate recovery values (e.g., 25 BPM and 26 BPM).

In FIG. 6L, device 600 displays details user interface 648c on display 602 in response to third input 646c in FIG. 6I. Details user interface 648c includes heart rate recovery value 650c, date 652c, source 654c, date added to health 656c, activity type 658c, recovery behavior 660c, medication factor 662c, and device details 664c, which are analogous to 650a-664a of details user interface 648a as described in detail with respect to FIG. 6J. In particular, heart rate recovery value 650c is “25 BPM,” the same as heart rate recovery value 650a of FIG. 6J. The physical activities in which heart rate data was collected for use in determining heart recovery value 650c are indicated by activity type 658c (e.g., “HIKING”) and recovery behavior 660c (e.g., “YOGA”). Activity type 658c and recovery behavior 660c are different from activity type 658a (e.g., “OUTDOOR RUN”) and recovery behavior 660a (e.g., “OUTDOOR WALK”) of FIG. 6J. Despite being generated using different activity types (e.g., 658a; 658c) and recovery behaviors (e.g., 660a; 660c), heart rate recovery value 650c of FIG. 6L and heart rate recovery value 650a of FIG. 6J are the same, reading “25 BPM.” In some embodiments, different activity types (e.g., 658a; 658c) and different recovery types (e.g., 660a; 660c) result in different heart rate recovery values (e.g., 25 BPM and 26 BPM). In some embodiments, the same activity types (e.g., hiking) and different recovery types (e.g., outdoor walk; yoga) result in the same heart rate recovery values (e.g., 25 BPM). In some embodiments, the same activity types (e.g., hiking) and different recovery types (e.g., outdoor walk; yoga) result in different heart rate recovery values (e.g., 25 BPM and 26 BPM). In some embodiments, different activity types (e.g., outdoor run; hiking) and the same recovery type (e.g., outdoor walk) result in the same heart rate recovery values (e.g., 25 BPM). In some embodiments, different activity types (e.g., outdoor run; hiking) and the same recovery type (e.g., outdoor walk) result in different heart rate recovery values (e.g., 25 BPM and 26 BPM).

In FIG. 6M, device 600 displays details user interface 648d on display 602 in response to fourth input 646d in FIG. 6I. Details user interface 648d includes heart rate recovery value 650d, date 652d, source 654d, date added to health 656d, activity type 658d, recovery behavior 660d, medication factor 662d, and device details 664d, which are analogous to 650a-664a of details user interface 648a as described in detail with respect to FIG. 6J. In particular, heart rate recovery value 650d is “25 BPM,” the same as heart rate recovery value 650a of FIG. 6J. The physical activities in which heart rate data was collected for use in determining heart recovery value 650d are indicated by activity type 658d (e.g., “OUTDOOR RUN”) and recovery behavior 660d (e.g., “OUTDOOR WALK”). Activity type 658d and recovery behavior 660d are the same as activity type 658a and recovery behavior 660a of FIG. 6J, an “OUTDOOR RUN” and an “OUTDOOR WALK,” respectively. As indicated by “ALBUTEROL INHALER” in medication factor 662d, heart rate recovery value 650d was generated after use of a medication that affects heart rate, different from “NO” medication in medication factor 662a of FIG. 6J. Despite being generated with different medication factors, heart rate recovery value 650d of FIG. 6M and heart rate recovery value 650a of FIG. 6J are the same, reading “25 BPM.” In some embodiments, the same activity type (e.g., 658a; 658b) and same recovery type (e.g., 660a; 660b) recorded with different medication factors (e.g., 662a; 662d) result in different heart rate recovery values (e.g., 25 BPM and 26 BPM). In some embodiments, different activity types (e.g., outdoor run; hiking) and/or recovery types (e.g., outdoor walk; yoga) with different medication factors (e.g., 662a; 662d) result in the same heart rate recovery values (e.g., 25 BPM). In some embodiments, different activity types (e.g., outdoor run; hiking) and/or recovery types (e.g., outdoor walk; yoga) with different medication factors (e.g., 662a; 662d) result in different heart rate recovery values (e.g., 25 BPM and 26 BPM).

FIG. 7 is a flow diagram illustrating a method for displaying user interfaces including information related to physiological measurements using a computer system in accordance with some embodiments. Method 700 is performed at a computer system (e.g., 100, 300, 500, 600, 604) (e.g., a smartphone, a desktop computer, a laptop, a tablet, or a head mounted device (e.g., a head mounted augmented reality and/or extended reality device)) that is in communication with a display generation component (e.g., a display controller, a touch-sensitive display system, and/or a head mounted display system) and one or more sensors (e.g., a heart rate sensor, an optical heart sensor, an electrical heart sensor, blood flow sensors, an accelerometer, and/or a gyroscope; an integrated sensor; a sensor in communication with the computer system (e.g., a sensor integrated into a connected external device)). Some operations in method 700 are, optionally, combined, the orders of some operations are, optionally, changed, and some operations are, optionally, omitted.

As described below, method 700 provides an intuitive way for displaying user interfaces including information related to physiological measurements. The method reduces the cognitive burden on a user for viewing user interfaces including information related to physiological measurements, thereby creating a more efficient human-machine interface. For battery-operated computing devices, enabling a user to view user interfaces including information related to physiological measurements faster and more efficiently conserves power and increases the time between battery charges.

While a user (e.g., a user wearing the computer system (such as on a wrist or on an arm) and/or a user holding the computer system) of the computer system (e.g., 600 and/or 604) performs a first activity (e.g., “OUTDOOR RUN” of FIGS. 6A-6B) (e.g., running, walking, and/or hiking), the computer system (e.g., 600 and/or 604) detects (702), via the one or more sensors, that a physiological parameter (e.g., as indicated by 610 and/or 610a) (e.g., heart rate or blood pressure) of the user of the computer system is at a first level (e.g., as indicated by 610a) (e.g., heart rate of 165 bpm, 170 bpm, or 175 bpm; or a systolic blood pressure of 190, 200, or 210). In some embodiments, the computer system identifies the first activity based on a workout tracking function (e.g., an activity identification function) and/or based on one or more user inputs identifying the activity (e.g., via selection of a specific workout type (e.g., jog, swim, biking)).

Subsequent to (e.g., after a predetermined amount of time, such as 1 minute, 2 minutes, or 3 minutes) detecting the physiological parameter at the first level and while the user of the computer system performs a second activity (e.g., “OUTDOOR WALK” of FIGS. 6E-6F) (e.g., walking, climbing steps, and/or without ceasing exercising) that is different from the first activity (e.g., “OUTDOOR RUN” of FIGS. 6A-6B), the computer system (e.g., 600 and/or 604) detects (704), via the one or more sensors (e.g., 534, 536, 532, heart rate sensor, and/or blood oxygen sensor), that the physiological parameter (e.g., heart rate or blood pressure) of the user of the computer system is at a second level (e.g., as indicated by 610b and/or 610c) (e.g., heart rate of 130 bpm, 135 bpm, or 140 bpm; or a systolic blood pressure of 160, 170, or 180) that is different from the first level (e.g., 610a) (e.g., second level is lower than the first level).

The computer system (e.g., 600 and/or 604) displays (706), via the display generation component (e.g., 602 and/or 606) and based on the first level (e.g., 610a) and the second level (e.g., 610b and/or 610c), a predictive change in the physiological parameter (e.g., 628d and/or 630e) (e.g., different from the actual change in the physiological parameter between detecting the first level and detecting the second level (e.g., a predictive change from the first level)) had the second activity (e.g., “OUTDOOR WALK” of FIGS. 6E-6F) (e.g., walking, climbing steps, and/or other exercise) been a third activity (e.g., ceasing to exercise and/or resting) that is different from the second activity. In some embodiments, the third activity is also different from the first activity. In some embodiments, the predictive change in the physiological parameter is based on the duration of time elapsed between detecting the physiological parameter of the first level and detecting the physiological parameter of the second level. In some embodiments, the predictive change in the physiological parameter predicts what the change (e.g., reduction or increase) between a first measurement (resulting in the first level) and a second measurement (resulting in the second level) would have been had the user been performing the third activity, rather than the second activity. In some embodiments, the computer system determines the predictive change in the physiological parameter before the predictive change in the physiological parameter is displayed. Displaying a predictive change in the physiological parameter had the second activity been the third activity provides the user with feedback about the information (e.g., levels of the physiological parameter) the computer system has detected about the user and provides the user with feedback about what the level of the change in the physiological parameter would have been had the user performed the third activity, without requiring the user to perform the third activity, thereby providing the user with improved visual feedback.

In some embodiments, the physiological parameter is a heart rate (e.g., as indicated by 610, 610a, 610b, 610c, 624e, 626e, 628a-628e, and/or 630b-630e). In some embodiments, the computer system uses a heart rate sensor to measure the heart rate of the user during the first and second activities. In some embodiments, the heart rate sensor is wirelessly connected to the computer system (e.g., the heart rate sensor is worn around a wrist of the user, the heart rate sensor is worn around a chest of the user). Displaying a predictive change in heart rate had the second activity been the third activity provides the user with feedback about what the computer system is sensing about the user's heart rate, thereby providing the user with improved visual feedback.

In some embodiments, the predictive change in the physiological parameter is a predicted heart rate recovery (e.g., 628d, 630e, 640a-640g, 644a-644j, and/or 650a-650d). In some embodiments, heart rate recovery is the decrease of heart rate at 1 minute after cessation of exercise. In some embodiments, heart rate recovery is a measure of how quickly your heart rate goes down after intense exercise. In some embodiments, heart rate recovery is the decrease in the number of heart beats per minute one minute after ceasing to workout. In some embodiments, the predicted heart rate recovery is a determined prediction of the user's heart rate recovery. Displaying a predicted heart rate recover provides the user with feedback about what the computer system is sensing about the user's heart rate and gives the user an indication of their health without requiring the user to perform specific activities (e.g., strenuous activity followed by no activity), thereby providing the user with improved visual feedback.

In some embodiments, the physiological parameter (e.g., heart rate or blood pressure) of the user at the second level (e.g., 628f) is detected after a predetermined amount of time (e.g., 30 seconds, 1 minute, or 2 minutes) after detecting that the first activity has ceased (e.g., 634) (e.g., has ended or is no longer being detected). In some embodiments, the predetermined period of time is the same as the time for which the change in the physiological parameter is predicted (e.g., the second level is measured one minute after the computer system detects the end of the first activity and the predicted change in the physiological parameter is a predicted change over 1 minute). In some embodiments, detecting that the first activity has ceased includes and/or is based on detecting an input to stop a workout tracking function associated with the first activity (e.g., stopping a workout function). Displaying a predictive change in the physiological parameter provides the user with feedback about what the computer system is sensing about the user's after a predetermined period of time after the user ends the first activity, thereby providing the user with improved visual feedback.

In some embodiments, the first activity includes a first exercise (e.g., 658a and/or 658c) (e.g., running and/or not ceasing activity or exercise) and the second activity includes a second exercise (e.g., 660a and/or 660c) different from the first exercise (e.g., a cool down walk and/or not ceasing activity or exercise). Displaying a predictive change in the physiological parameter even when both the first activity and the second activity include exercising provides the user with a standardized measure of the user's health without requiring the user to perform specific activities (e.g., strenuous activity followed by no activity), thereby providing the user with improved visual feedback.

In some embodiments, the third activity does not include exercising (e.g., the user has stopped exercising and/or is sitting or standing). Displaying a predictive change in the physiological parameter even the third activity does not include exercising provides the user with a standardized measure of the user's health without requiring the user to perform specific activities (e.g., strenuous activity followed by no activity), thereby providing the user with improved visual feedback.

In some embodiments, subsequent to (e.g., the next day or the next week) detecting that the physiological parameter of the user of the computer system is at the first level and the second level (and, optionally, subsequent to displaying the predictive change in the physiological parameter): while the user of the computer system performs (e.g., performs on a subsequent day or week) the first activity (e.g., “OUTDOOR RUN” of FIGS. 6A-6B) (e.g., running, walking, and/or hiking), computer system (e.g., 600 and/or 604) detects, via the one or more sensors, that the physiological parameter (e.g., heart rate or blood pressure) of the user of the computer system is at a third level (e.g., heart rate of 165 bpm, 170 bpm, or 175 bpm; or a systolic blood pressure of 190, 200, or 210) that is different from the first level.

In some embodiments, subsequent to (e.g., the next day or the next week) detecting that the physiological parameter of the user of the computer system is at the first level and the second level (and, optionally, subsequent to displaying the predictive change in the physiological parameter) and subsequent to (e.g., after a predetermined amount of time, such as 1 minute, 2 minutes, or 3 minutes) detecting the physiological parameter at the third level and while the user of the computer system performs the second activity (e.g., “OUTDOOR WALK” of FIGS. 6E-6F) (e.g., walking, climbing steps, and/or without ceasing exercising) that is different from the first activity, computer system (e.g., 600 and/or 604) detects, via the one or more sensors, that the physiological parameter (e.g., heart rate or blood pressure) of the user of the computer system is at a fourth level (e.g., heart rate of 130 bpm, 135 bpm, or 140 bpm; or a systolic blood pressure of 160, 170, or 180) that is different from the third level (e.g., fourth level is lower than the second level).

In some embodiments, subsequent to (e.g., the next day or the next week) detecting that the physiological parameter of the user of the computer system is at the first level and the second level (and, optionally, subsequent to displaying the predictive change in the physiological parameter): the computer system (e.g., 600, 604) displays, via the display generation component (e.g., 602 and/or 606) and based on the third level and the fourth level, a second predictive change in the physiological parameter (e.g., 640a-640g, 644a-644j, and/or 650a-650d) (e.g., different from the actual change in the physiological parameter between detecting the first level and detecting the second level (e.g., a predictive change from the first level)) had the second activity (e.g., walking, climbing steps, and/or other exercise) been the third activity (e.g., ceasing to exercise and/or resting) that is different from the second activity, wherein the second predictive change in the physiological parameter is the same as the predictive change in the physiological parameter (e.g., 650a-650d). In some embodiments, the computer system determines and displays the same predictive change in the physiological parameter when the user performs the same activities on multiple days, even when the difference between the first and second levels is different from the difference between the third and fourth levels. Displaying the same predictive change in the physiological parameter after performing the same workouts at a different time provides the user with visual feedback about a standardized measure of the user's health without requiring the user to perform specific activities (e.g., strenuous activity followed by no activity), thereby providing the user with improved visual feedback.

In some embodiments, subsequent to (e.g., the next day or the next week) detecting that the physiological parameter of the user of the computer system is at the first level and the second level (and, optionally, subsequent to displaying the predictive change in the physiological parameter): while the user of the computer system performs a fourth activity (e.g., 658c) (e.g., running, walking, and/or hiking) (e.g., performs on a subsequent day or week) that is different from the first activity, the computer system (e.g., 600, 604) detects, via the one or more sensors, that the physiological parameter (e.g., heart rate or blood pressure) of the user of the computer system is at a fifth level (e.g., heart rate of 165 bpm, 170 bpm, or 175 bpm; or a systolic blood pressure of 190, 200, or 210) that is different from the first level.

In some embodiments, subsequent to (e.g., the next day or the next week) detecting that the physiological parameter of the user of the computer system is at the first level and the second level (and, optionally, subsequent to displaying the predictive change in the physiological parameter) and subsequent to (e.g., after a predetermined amount of time, such as 1 minute, 2 minutes, or 3 minutes) detecting the physiological parameter at the fifth level and while the user of the computer system performs a fifth activity (e.g., 660c) (e.g., walking, climbing steps, and/or without ceasing exercising) that is different from the fourth activity and the second activity, the computer system (e.g., 600 and/or 604) detects, via the one or more sensors, that the physiological parameter (e.g., heart rate or blood pressure) of the user of the computer system is at a sixth level (e.g., heart rate of 130 bpm, 135 bpm, or 140 bpm; or a systolic blood pressure of 160, 170, or 180) that is different from the fifth level (e.g., sixth level is lower than the fifth level).

In some embodiments, subsequent to (e.g., the next day or the next week) detecting that the physiological parameter of the user of the computer system is at the first level and the second level (and, optionally, subsequent to displaying the predictive change in the physiological parameter): the computer system (e.g., 600 and/or 604) displays, via the display generation component (e.g., 602 and/or 606) and based on the fifth level and the sixth level, a third predictive change in the physiological parameter (e.g., 650c) (e.g., different from the actual change in the physiological parameter between detecting the first level and detecting the second level (e.g., a predictive change from the first level)) had the fifth activity (e.g., walking, climbing steps, and/or other exercise) been the third activity (e.g., ceasing to exercise and/or resting) that is different from the fifth activity, wherein the third predictive change in the physiological parameter is the same as the predictive change in the physiological parameter. In some embodiments, the computer system determines and displays the same predictive change in the physiological parameter when the user performs the same activities on multiple days, even when the difference between the first and second levels is different from the difference between the third and fourth levels. Displaying the same predictive change in the physiological parameter after the user performs different workouts provides the user with visual feedback about a standardized measure of the user's health without requiring the user to perform specific activities (e.g., strenuous activity followed by no activity), thereby providing the user with improved visual feedback.

In some embodiments, wherein the computer system is in communication with one or more input devices (e.g., a touch sensitive surface, a keyboard, and/or a mouse), while displaying (e.g., on display 602 and/or 606) the predictive change in the physiological parameter (e.g., 630e), the computer system (e.g., 600, 604) receives, via the one or more input devices (e.g., 602), a set of one or more inputs (e.g., 636) that includes a selection of the predictive change in the physiological parameter (e.g., 630e).

In some embodiments, wherein the computer system is in communication with one or more input devices (e.g., a touch sensitive surface, a keyboard, and/or a mouse), in response to receiving selection (e.g., 636) of the predictive change in the physiological parameter (e.g., 630e), the computer system (e.g., 600 and/or 604) displays, via the display generation component, a graph (e.g., 640) that includes an indication of a plurality (e.g., 3, 5, or 7) of respective predictive changes in the physiological parameter (e.g., 640a-640g). In some embodiments, the computer system predicts the change in the physiological parameter had the user performed the third activity (e.g., not exercising and/or resting) as the latter activity over multiple days and displays a graph that includes the predicted change in the physiological parameter for those days. Displaying a graph that includes indication of a plurality of respective predictive changes in the physiological parameter provides the user with visual feedback about a standardized measure of the user's health over time, thereby providing the user with improved visual feedback.

In some embodiments, the predictive change in the physiological parameter had the second activity been the third activity is based on one or more medications the user is taking (e.g., 662d). In some embodiments, the computer system receives user input indicating the medication (e.g., type, quantity, frequency, and/or duration) that the user is taking and uses properties of the medication to determine the predictive change (e.g., determines what the user's heart rate recovery would be had the user not been using the medication). In some embodiments, the computer system takes into account the one or more medications the user is taking (e.g., as entered into the computer system or a remote database by the user) when determining the predictive change in the physiological parameter. In some embodiments, an algorithm is used to determine the predictive change in the physiological parameter and the algorithm includes, as input, information relating to the one or more medications the user is taking. Providing the user with the predictive change in the physiological parameter based on one or more medications the user is taking provides the user with visual feedback about a standardized measure of the user's health taking into account the effects of the medications, thereby providing the user with improved visual feedback.

In some embodiments, the computer system (e.g., 600 and/or 604) displays, via the display generation component (e.g., 602 and/or 606) and concurrently with the predictive change in the physiological parameter (e.g., 650a-650d), indications (e.g., textual and/or graphical indications) of the first activity (e.g., 658a-658d) and the second activity (e.g., 660a-660d). Concurrently displaying the predictive change in the physiological parameter with the indications of the first activity and the second activity provides the user with visual feedback about the activities upon which the predictive change in the physiological parameter is based, thereby providing the user with improved visual feedback.

In some embodiments, the computer system (e.g., 600, 604) displays the predictive change in the physiological parameter without displaying an actual change in the physiological parameter between the first level and the second level (e.g., the difference between the first level and the second level is not displayed concurrently with the predictive change in the physiological parameter). Displaying the predictive change in the physiological parameter without displaying the actual change in the physiological parameter between the first level and the second level reduces visual clutter in the user interface and enables the user to more quickly and easily access the predictive change in the physiological parameter, thereby providing the user with improved visual feedback.

In some embodiments, the predictive change in the physiological parameter is based on one or more medications the user is taking (e.g., 662a-662d). In some embodiments, the computers system (e.g., 600 and/or 604) displays, via the display generation component (e.g., 602 and/or 606) and concurrently with the predictive change in the physiological parameter (e.g., 650d), one or more indications of the one or more medications the user is taking (e.g., 662d). Displaying one or more indications of the one or more medications the user is taking that have affected the predictive change in the physiological parameter provides the user with visual feedback about what information the computer system is using to determine the predictive change in the physiological parameter, thereby providing the user with improved visual feedback.

In some embodiments, the predictive change in the physiological parameter (e.g., 630e) is higher than an actual change between the first level and the second level (e.g., 20 BPM based on 624e and 626e of FIG. 6F). Displaying a predictive change in the physiological parameter that is higher than the actual change between the first level and the second level enables the computer system to provide the user with visual feedback about a standardized measure of the user's health without requiring the user to perform specific activities (e.g., strenuous activity followed by no activity), thereby providing the user with improved visual feedback

In some embodiments, the computer system (e.g., 600, 604) displays, via the display generation component (e.g., 602 and/or 606) (e.g., and, optionally, concurrently with the predictive change in the physiological parameter), an indication (e.g., binary indication and/or quantitative indication) of whether a trend (e.g., 640i) among a plurality of predictive changes in the physiological parameter are positive or negative. Displaying an indication of whether a trend among the plurality of predictive changes in the physiological parameter are positive or negative provides the user with feedback about whether their health is increasing or decreasing, thereby providing the user with improved visual feedback.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

As described above, one aspect of the present technology is the gathering and use of data available from various sources to allow users to view and manage relevant clinical, health-related, and/or physiological data. The present disclosure contemplates that in some instances, this gathered data may include personal information data that uniquely identifies or can be used to contact or locate a specific person. Such personal information data can include demographic data, location-based data, telephone numbers, email addresses, social network IDs, home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to display user-specific clinical, health-related, or physiological measurements data. Accordingly, use of such personal information data enables users to view and manage their corresponding data. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of clinical, health-related, or physiological measurements data, the present technology can be configured to allow users to select to “opt in” or “opt out” of participation in the collection of personal information data during registration for services or anytime thereafter. In another example, users can select not to provide clinical, health-related, or physiological measurements data for targeted content delivery services. In yet another example, users can select to limit the length of time clinical, health-related, or physiological measurements data is maintained or entirely prohibit the development of a baseline mood profile. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, clinical, health-related, or physiological measurements data can be displayed to users by inferring preferences based on non-personal information data or a bare minimum amount of personal information, such as the content being requested by the device associated with a user, other non-personal information available to the computer system, or publicly available information.

What is claimed is:

1. A computer system configured to communicate with a display generation component and one or more sensors, comprising:
one or more processors; and
memory storing one or more programs configured to be executed by the one or more processors, the one or more programs including instructions for:
while a user of the computer system performs a first activity, detecting, via the one or more sensors, that a physiological parameter of the user of the computer system is at a first level;
subsequent to detecting the physiological parameter at the first level and while the user of the computer system performs a second activity that is different from the first activity, detecting, via the one or more sensors, that the physiological parameter of the user of the computer system is at a second level that is different from the first level; and
displaying, via the display generation component and based on the first level and the second level, a predictive change in the physiological parameter had the second activity been a third activity that is different from the second activity.

2. The computer system of claim 1, wherein the physiological parameter is a heart rate.

3. The computer system of claim 1, wherein the predictive change in the physiological parameter is a predicted heart rate recovery.

4. The computer system of claim 1, wherein the physiological parameter of the user at the second level is detected after a predetermined amount of time after detecting that the first activity has ceased.

5. The computer system of claim 1, wherein the first activity includes a first exercise and the second activity includes a second exercise different from the first exercise.

6. The computer system of claim 1, wherein the third activity does not include exercising.

7. The computer system of claim 1, the one or more programs further including instructions for:
subsequent to detecting that the physiological parameter of the user of the computer system is at the first level and the second level:
while the user of the computer system performs the first activity, detecting, via the one or more sensors, that the physiological parameter of the user of the computer system is at a third level that is different from the first level;
subsequent to detecting the physiological parameter at the third level and while the user of the computer system performs the second activity that is different from the first activity, detecting, via the one or more sensors, that the physiological parameter of the user of the computer system is at a fourth level that is different from the third level; and
displaying, via the display generation component and based on the third level and the fourth level, a second predictive change in the physiological parameter had the second activity been the third activity that is different from the second activity, wherein the second predictive change in the physiological parameter is the same as the predictive change in the physiological parameter.

8. The computer system of claim 1, the one or more programs further including instructions for:
subsequent to detecting that the physiological parameter of the user of the computer system is at the first level and the second level:
while the user of the computer system performs a fourth activity that is different from the first activity, detecting, via the one or more sensors, that the physiological parameter of the user of the computer system is at a fifth level that is different from the first level;
subsequent to detecting the physiological parameter at the fifth level and while the user of the computer system performs a fifth activity that is different from the fourth activity and the second activity, detecting, via the one or more sensors, that the physiological parameter of the user of the computer system is at a sixth level that is different from the fifth level; and
displaying, via the display generation component and based on the fifth level and the sixth level, a third predictive change in the physiological parameter had the fifth activity been the third activity that is different from the fifth activity, wherein the third predictive change in the physiological parameter is the same as the predictive change in the physiological parameter.

9. The computer system of claim 1, wherein the computer system is in communication with one or more input devices, the one or more programs further including instructions for:
while displaying the predictive change in the physiological parameter, receiving, via the one or more input devices, a set of one or more inputs that includes a selection of the predictive change in the physiological parameter; and
in response to receiving selection of the predictive change in the physiological parameter, displaying, via the display generation component, a graph that includes an indication of a plurality of respective predictive changes in the physiological parameter.

10. The computer system of claim 1, wherein the predictive change in the physiological parameter had the second activity been the third activity is based on one or more medications the user is taking.

11. The computer system of claim 1, the one or more programs further including instructions for:
displaying, via the display generation component and concurrently with the predictive change in the physiological parameter, indications of the first activity and the second activity.

12. The computer system of claim 1, wherein the computer system displays the predictive change in the physiological parameter without displaying an actual change in the physiological parameter between the first level and the second level.

13. The computer system of claim 1, wherein the predictive change in the physiological parameter is based on one or more medications the user is taking, the one or more programs further including instructions for:
displaying, via the display generation component and concurrently with the predictive change in the physiological parameter, one or more indications of the one or more medications the user is taking.

14. The computer system of claim 1, wherein the predictive change in the physiological parameter is higher than an actual change between the first level and the second level.

15. The computer system of claim 1, the one or more programs further including instructions for:
displaying, via the display generation component, an indication of whether a trend among a plurality of predictive changes in the physiological parameter are positive or negative.

16. A non-transitory computer-readable storage medium storing one or more programs configured to be executed by one or more processors of a computer system that is in communication with a display generation component and one or more sensors, the one or more programs including instructions for:
while a user of the computer system performs a first activity, detecting, via the one or more sensors, that a physiological parameter of the user of the computer system is at a first level;
subsequent to detecting the physiological parameter at the first level and while the user of the computer system performs a second activity that is different from the first activity, detecting, via the one or more sensors, that the physiological parameter of the user of the computer system is at a second level that is different from the first level; and
displaying, via the display generation component and based on the first level and the second level, a predictive change in the physiological parameter had the second activity been a third activity that is different from the second activity.

17. A method, comprising:
at a computer system that is in communication with a display generation component and one or more sensors;
while a user of the computer system performs a first activity, detecting, via the one or more sensors, that a physiological parameter of the user of the computer system is at a first level;
subsequent to detecting the physiological parameter at the first level and while the user of the computer system performs a second activity that is different from the first activity, detecting, via the one or more sensors, that the physiological parameter of the user of the computer system is at a second level that is different from the first level; and
displaying, via the display generation component and based on the first level and the second level, a predictive change in the physiological parameter had the second activity been a third activity that is different from the second activity.
